(19) United States
(12) Patent Application Publication
(10) Pub. No.: US 2002/0151515 A1
(43) Pub. Date: Oct. 17, 2002

(54) Preparation and use of superior vaccines

(76) Inventor: Bruce L. Roberts, Southboro, MA (US)

Correspondence Address: Genzyme Corporation, 15 Pleasant Street Connector, P.O. Box 9322, Framingham, MA, 01701-9322, US

(21) Appl. No.: 10/033,145

(22) Filed: Nov. 5, 2001

Related U.S. Application Data
(63) Continuation of application No. PCT/US99/13800, filed on Jun. 18, 1999.

(51) Int. Cl. A61K 48/00; C12Q 1/68; C07H 21/04; C12P 21/02; C12N 5/06; C12N 5/08

(52) U.S. Cl. 514/44; 536/23.2; 435/366; 435/325; 435/320.1; 435/6; 435/287.2

(57) ABSTRACT

This invention provides an isolated population of polynucleotides comprising or corresponding to at least one polynucleotide shown in Table 1 and their respective complements. It also provides a polynucleotide encoding a ligand or antibody or engineered protein that binds to a cell surface protein of an antigen presenting cell and wherein the polynucleotide comprises or corresponds to a polynucleotide shown in Table 1 or its complement. The invention further provides a polynucleotide that encodes a transcription factor and wherein the polynucleotide comprises or corresponds to a polynucleotide shown in Table 1 or its complement.

### TECHNICAL FIELD

The present invention is directed to enhanced immunotherapy of human malignancies such as cancers.

### BACKGROUND

The complex relationships between the immune system and tumor cells during the course of their pathogenesis have not been thoroughly understood. However, the mere fact that a host immune system has the potential to recognize and eventually eradicate tumor cells has warranted immunotherapy as one of the most promising approaches for cancer treatment. Most tumors express altered or abnormal gene products as the result of uncontrolled cell growth and malignant transformation. These abnormal gene products are often antigenic to the host immune system, rendering the tumor cells potentially susceptible to immunocytolysis. Gilboa et al. (1998) Cancer 1 mm. Immunother. 46:82-87.

Cytotoxic T lymphocyte (CTL)-mediated cellular immunity is regarded as an important weapon for a host defense system against many tumors. A variety of molecular factors determine whether a tumor cell can be recognized by the host immune system and eventually lysed by CTL. Lindauer et al. (1998) J. Mol. Med. 76.32-47. Tumor associated antigens are proteolytically degraded into small peptide epitopes that compete for binding to and presentation by a finite number of major histocompatibility complex (MHC) molecules. The formed MHC-peptide complexes can be recognized by naive CTLs via their T cell receptors. Further activation of the naive CTLs requires functions of costimulatory factors, most of which are associated with professional antigen presenting cells (APCs). The activated antigen-specific CTLs then differentiate into cytolytic effector cells that are capable of lysing tumor cells bearing specific tumor antigens.

Various tumor associated antigens have been identified so far. with the vast majority being melonoma-related. Lindauer et al. supra. Tumor associated antigens can be categorized into four classes: the differentiation antigens which are normal proteins over-expressed by tumor cells such as gplO0; viral antigens such as HPV16 E6 and E7; the cancer/testes family of antigens typified by MACE; and the mutated proteins such as ras or p53. All these tumor antigens, when processed properly and presented favorably by MHC molecules, can be the targets for recognition and binding by T cell receptors.

However, mere presentation of the tumor antigen via MHC and subsequent recognition by T cell receptors are not insufficient to activate a robust cytotoxic immune response that can lyse the tumor cells. Many co-factors having immunostimulatory functions are necessary for efficient CTL activation. Indeed, binding and stimulating T cell receptors in the absence of these costimulatory factors may cause the T cells to be unresponsive to further antigenic stimulation, which is an anergy state potentially responsible for immune tolerance to many tumor self antigens.

Costimulatory functions have primarily been associated with professional antigen-presenting cells (APC). For example, upon exposure to specific signals such as inflammatory agents, APCs have the capacity to up-regulate T cell proliferation and IL-2 production, which are necessary processes for CTL activation. APCs are also known for secreting T cell growth factors to amplify antigen-specific CTL response. In addition, activated APCs can provide a favorable lymphoid environment for antigen presentation and CTL activation, either by secreting chemokines that can induce the migration of immune effector cells to antigen presenting sites; or by migrating to T cell rich sites such as draining lymph nodes where favorable APC:T cell interactions can occur.

Various gene based vaccines have been used to deliver transgenes encoding tumor antigen to APCs in vivo for antigen presentation, but very few are proven effective to elicite anti-tumor immunoactivity. While these gene based vaccines, including genetically modified APCs, may be efficient for antigen presentation, they do not provide any modulation of the functional state of the endogenous APCs such as stimulating/facilitating the activation of T-cells. The present invention addresses this limitation and provides an enhanced vaccine composition for eliciting effective anti-tumor immune responses.

### DISCLOSURE OF THE INVENTION

This invention provides an isolated population of polynucleotides comprising or corresponding to at least one polynucleotide shown in Table 1 and their respective complements. It also provides a polynucleotide encoding a ligand or antibody or engineered protein that binds to a cell surface protein of an antigen presenting cell and wherein the polynucleotide comprises or corresponds to a polynucleotide shown in Table 1 or its complement. The invention further provides a polynucleotide that encodes a transcription factor and wherein the polynucleotide comprises or corresponds to a polynucleotide shown in Table 1 or its complement.

Further provided herein is a polynucleotide comprising a first polynucleotide comprising encoding an immunostimulatory factor that is differentially expressed in an antigen presenting cell and comprising or corresponding to a tag shown in Table 1 or its complement. In one embodiment, the first polynucleotide encodes a factor selected from the group consisting of PARC, TARC, monocyte chemoattractant protein-4 (MDP-4), MDC, escalectin, MCP-2 or a biologically active fragments thereof. The polynucleotides can further comprise a first and second promoter, wherein the first and second polynucleotides are under the transcriptional control of the first and second promoters, respectively.

Also provided by this invention is a polynucleotide comprising a first polynucleotide comprising encoding an immunostimulatory factor that is differentially expressed in an antigen presenting cell that is differentially expressed in an antigen presenting cell and comprising or corresponding to a tag shown in Table 1 and second polynucleotide that modulates the expression of the first polynucleotide. Further provided is a polynucleotide comprising a first polynucleotide encoding an antigen and a second polynucleotide that modulates the expression of a third polynucleotide which encodes an immunstimulatory factor that is differentially expressed in an antigen presenting cell, wherein the third polynucleotide comprises or corresponds to a tag shown in Table 1. The first polynucleotide may encode PARC, monocyte chemoattractant protein-4 (MDP-4), MDC, escalectin, MCP-2 or a biologically active fragments thereof. Also provided herein is a polynucleotide comprising a first polynucleotide encoding an engineered protein or polypeptide that binds to a cell surface protein of antigen presenting cells thereby modulating either directly or indirectly by a signal transduction pathway and a second polynucleotide encoding an immunostimulatory factor comprising or corresponding to a tag shown in Table 1. Promoters can be operatively linked to the polynucleotides to direct expression thereof.

The polynucleotides can be inserted within a gene delivery vehicle or a host cell. Alternatively they can be attached to a chip or within a database for computational analysis.

The compositions of this invention are useful to induce an immune response in a subject. They also are useful to modulate the genotype of an antigen presenting and to screen for a candidate therapeutic agent that modulates the expression of a polynucleotide differentially expressed in an antigen.

### BRIEF DESCRIPTION OF THE TABLE

The Table depicts a series of mRNA sequences, identified by SAGE analysis. Tags isolated from various populations were isolated and analyzed: tags expressed in monocytes; tags expressed in monocyte-derived immature dendritic cells; and tags expressed in monocyte-derived mature dendritic cells, that have been stimulated to mature with TNFα. The columns of the tables are as follows: “Accession” is the accession number for the EST in the public databases; “tag” is the 10mer SAGE tag; “Seq. ID No.” is the corresponding Sequence ID number for the tag found at the end of the specification and claims. The description identifies the known gene or EST that corresponds to a tag. If the description section is blank or contains “NM” that identifies a novel tag as no match was found.

### MODE(S) FOR CARRYING OUT THE INVENTION

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

The practice of the present invention employs, unless otherwise indicated, conventional techniques of molecular biology (including recombinant techniques), microbiology, cell biology, biochemistry and immunology, which are within the skill of the art. Such techniques are explained fully in the literature. These methods are described in the following publications. See, e.g. Sambrook et al. MOLECULAR CLONING: A LABORATORY MANUAL, 2^{nd} edition (1989); CURRENT PROTOCOLS IN MOLECULAR BIOLOGY (F. M. Ausubel et al. eds. (1987)); the series METHODS IN ENZYMOLOGY (Academic Press, Inc.); “PCR: A PRACTICAL APPROACH” (M. MacPherson et al. IRL Press at Oxford University Press (1991)); PCR 2: A PRACTICAL APPROACH (M. J. MacPherson, B. D. Hames and G. R. Taylor eds. (1995)); ANTIBODIES, A LABORATORY MANUAL (Harlow and Lane eds. (1988)); and ANIMAL CELL CULTURE (R. I. Freshney ed. (1987)).

As used herein, certain terms may have the following defined meanings.

The singular form “a”, “an” and “the” include plural references unless the context clearly dictates otherwise. For example, the term “a cell” includes a plurality of cells, including mixtures thereof.

The term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline. preservatives, and the like. “Consisting of” shall mean excluding more than trace elements of other ingredients and substantial method steps for administering the compositions of this invention. Embodiments defined by each of these transition terms are within the scope of this invention.

The terms “polynucleotide” and “nucleic acid molecule” are used interchangeably to refer to polymeric forms of nucleotides of any length. The polynucleotides may contain deoxyribonucleotides, ribonucleotides, and/or their analogs. Nucleotides may have any three-dimensional structure, and may perform any function, known or unknown. The term “polynucleotide” includes, for example, single-, double-stranded and triple helical molecules, a gene or gene fragment, exons, introns, mRNA, tRNA, rRNA, ribozyn es, cDNA, recombinant polynucleotides, branched polynucleotides, plasmids, vectors, isolated DNA of any sequence, isolated RNA of any sequence, nucleic acid probes, and primers. A nucleic acid molecule may also comprise modified nucleic acid molecules.

A “gene” refers to a polynuclec tide containing at least one open reading frame that is capable of encoding a particular polypeptide or protein after being transcribed and translated.

A “gene product” refers to the amino acid (e.g. peptide or polypeptide) generated when a gene is transcribed and translated.

As used herein a second polynucleotide “corresponds to” another (a first) polynucleotide if it is related to the first polynucleotide by any of the following relationships:

1) The second polynucleotide comprises the first polynucleotide and the second polynucleotide encodes a gene product.

2) The second polynucleotide is 5′ or 3′ to the first polynucleotide in cDNA, RNA, genomic DNA, or fragment of any of these polynucleotides. For example, a second polynucleotide may be a fragment of a gene that includes the first and second polynucleotides. The first and second polynucleotides are related in that they are components of the gene coding for a gene product, such as a protein or antibody. However, it is not necessary that the second polynucleotide comprises or overlaps with the first polynucleotide to be encompassed within the definition of “corresponding to” as used herein. For example, the first polynucleotide may be a fragment of a 3′ untranslated region of the second polynucleotide, e.g., it may comprise a promoter sequence for the gene comprising the tag. The first and second polynucleotide may be fragment of a gene coding for a gene product. The second polynucleotide may be an exon of the gene while the first polynucleotide may be an intron of the gene.

3) The second polynucleotide is the complement of the first polynucleotide.

A “foreign polynucleotide” is a DNA sequence that is foreign to the cell, vector or position therein, wherein it is placed.

A “sequence tag” or “tag” or “SAGE tag” is a short oligonucleotide containing defined nucleotide sequence that occurs in a certain position of a gene transcript. The length of a tag is generally under about 20 nucleotides, preferably between 9 to 15 nucleotides, and more preferably 10 nucleotides. The tag can be used to identify the corresponding transcript and gene from which it was transcribed A tag can further comprise exogenous nucleotide sequences to facilitate the identification and utility of the tag. Such auxiliary sequences include, but are not limited to, restriction endonuclease cleavage sites and well known primer sequences for sequencing and cloning.

The term “peptide” is used in its broadest sense to refer to a compound of two or more subunit amino acids, amino acid analogs, or peptidomimetics. The subunits may be linked by peptide bonds. In another embodiment, the subunit may be linked by other bonds, e.g. ester, ether, etc. As used herein the term “amino acid” refers to either natural and/or unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is commonly called a polypeptide or a protein.

The term “cDNAs” refers to complementary DNA, that is mRNA molecules present in a cell or organism made in to cDNA with an enzyme such as reverse transcriptase. A “cDNA library” is a collection of all of the mRNA molecules present in a cell or organism, all turned into cDNA molecules with the enzyme reverse transcriptase, then inserted into “vectors”.

A “probe” when used in the context of polynucleotide manipulation refers to an oligonucleotide that is provided as a reagent to detect a target potentially present in a sample of interest by hybridizing with the target. Usually, a probe will comprise a label or a means by which a label can be attached, either before or subsequent to the hybridization reaction. Suitable labels include, but are not limited to radioisotopes, fluorochromes, chemiluminescent compounds, dyes, and proteins, including enzymes.

A “primer” is a short polynucleotide, generally with a free 3′-OH group that binds to a target or “template” potentially present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or a “set of primers” consisting of an “upstream” and a “downstream” primer, and a catalyst of polymerization, such as a DNA polymerase, and typically a thermally-stable polymerase enzyme. Methods for PCR are well known in the art, and taught, for example in “PCR: A PRACTICAL APPROACH” (M. MacPherson et al. IRL Press at Oxford University Press (1991)). All processes of producing replicate copies of a polynucleotide, such as PCR or gene cloning, are collectively referred to herein as “replication.” A primer can also be used as a probe in hybridization reactions, such as Southern or Northern blot analyses. Sambrook et al, supra.

A “promoter” is a region on a DNA molecule to which an RNA polymerase binds and initiates transcription. In an operon, the promoter is usually located at the operator end, adjacent but external to the operator. The nucleotide sequence of the promoter determines both the nature of the enzyme that attaches to it and the rate of RNA synthesis.

The term “genetically modified” means containing and/or expressing a foreign gene or nucleic acid sequence which in turn, modifies the genotype or phenotype of the cell or its progeny.

As used herein, “expression” or “expressed” refers to the process by which polynucleotides are transcribed into mRNA or by which transcription is enhanced. In another embodiment, the RNA is translated into peptides, polypeptides, or proteins. If the polynucleotide is derived from genomic DNA, expression may include splicing of the mRNA, if an appropriate eukaryotic host is selected.

“Differentially expressed” as applied to a gene, refers to the differential production of the mRNA transcribed from the gene or the protein product encoded by the gene. A differentially expressed gene may be overexpressed or underexpressed as compared to the expression level of a normal or control cell. In one aspect, it refers to a differential that is 3 times, preferably 5 times, or preferably 10 times higher or lower than the expression level detected in a control sample. The term “differentially expressed” also refers to nucleotide sequences in a cell or tissue which are expressed where silent in a control cell or not expressed where expressed in a control cell.

A “native” or “natural” antigen is a polypeptide, protein or a fragment which contains an epitope, which has been isolated from a natural biological source, and which can specifically bind to an antigen receptor, in particular a T cell antigen receptor (TCR), in a subject. It also substances which are immunogenic, i.e., immunogens, as well as substances which induce immunological unresponsiveness, or anergy, i.e., anergens.

A “self-antigen” also referred to herein as a native or wild-type antigen is an antigenic peptide that induces little or no immune response in the subject due to self-tolerance to the antigen. An example of a self-antigen is the human melanoma antigen gp 100.

The term “tumor associated antigen” or “TAA” refers to an antigen that is associated with or specific to a tumor. Examples of known TAAs include gp100, MART and MAGE.

The term “lysing” refers to the action of rupturing the cell wall and/or cell membrane of a cell through cytotoxic T-cell lymphocyte (CTL)-mediated cellular immunity. In a preferred embodiment, the lysis of cells is done to release cellular constituents from the lysed cells. For purposes of the present invention. “cellular constituents” is meant any component found within a cell. Such components include, but are not limited to, proteins, lipoproteins, glycoproteins, lipids. carbohydrates, nucleic acids, steroids, prostaglandins, and combinations and complexes thereof. The components are also referred to as “endogenous antigens.”

A “gene delivery vehicle” is defined as any molecule that can carry inserted polynucleotides into a host cell. Examples of gene delivery vehicles are liposomes, viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

A “viral vector” is defined as a recombinantly produced virus or viral particle that comprises a polynucleotide to be delivered into a host cell, either in vivo, ex vivo or in vitro. Examples of viral vectors include retroviral vectors, adenovirus vectors, adeno-associated virus vectors and the like. In aspects where gene transfer is mediated by a retroviral vector, a vector construct refers to the polynucleotide comprising the retroviral genome or part thereof, and a therapeutic gene. As used herein, “retroviral mediated gene transfer” or “retroviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. As used herein, retroviral vector refers to a viral particle capable of introducing exogenous nucleic acid into a cell through a viral or viral-like entry mechanism.

The term “isolated” means separated from constituents, cellular and otherwise, in which the polynucleotide, peptide, polypeptide, protein, antibody, or fragments thereof, are normally associated with in nature. In addition, a “concentrated”, “separated” or “diluted” polynucleotide, peptide, polypeptide, protein, antibody, or fragments thereof, is distinguishable from its naturally occurring counterpart in that the concentration or number of molecules per volume is greater than “concentrated” or less than “separated” than that of its naturally occurring counterpart. Although not explicitly stated for each of the inventions disclosed herein, it is to be understood that all of the above embodiments for each of the compositions disclosed below and under the appropriate conditions, are provided by this invention. Thus, a non-naturally occurring polynucleotide is provided as a separate embodiment from the isolated naturally occurring polynucleotide. A protein produced in a bacterial cell is provided as a separate embodiment from the naturally occurring protein isolated from a eukaryotic cell in which it is produced in nature.

“Host cell” or “recipient cell” is intended to include any individual cell or cell culture which can be or have been recipients for vectors or the incorporation of exogenous nucleic acid molecules, polynucleotides and/or proteins. It also is intended to include progeny of a single cell, and the progeny may not necessarily be completely identical (in morphology or in genomic or total DNA complement) to the original parent cell due to natural, accidental, or deliberate mutation. The cells may be prokaryotic or eukaryotic, and include but are not limited to bacterial cells, yeast cells, animal cells, and mammalian cells, e.g., murine, rat, simian or human.

A “subject” is a vertebrate, preferably a mammal, more preferably a human. Mammals include, but are not limited to, murmes, simians, humans, farm animals, sport animals, and pets.

A “control” is an alternative subject or sample used in an experiment for comparison purpose. A control can be “positive” or “negative.” For example, where the purpose of the experiment is to determine a correlation of an altered expression level of a proto-oncogene with a particular type of cancer, it is generally preferable to use a positive control (a subject or a sample from a subject, carrying such alteration and exhibiting syndromes characteristic of that disease), and a negative control (a subject or a sample from a subject lacking the altered expression and clinical syndrome of that disease).

The terms “major histocompatibility complex” or “MHC” refers to a complex of genes encoding cell-surface molecules that are required for antigen presentation to T cells and for rapid graft rejection. The proteins encoded by the MHC complex are known as “MHC molecules” and are classified into class I and class II MHC molecules. Class I MHC molecules include membrane heterodimeric proteins made up of an a chain encoded in the MHC associated noncovalently with b2-microglobulin. Class I MHC molecules are expressed by nearly all nucleated cells and have been shown to function in antigen presentation to CD8⁺ T cells. Class I molecules include HLA-A, -B, and -C in humans. Class II MHC molecules also include membrane heterodimeric proteins consisting of noncovalently associated a and b chains. Class II MHC are known to participate in antigen presentation to CD4⁺ T cells and, in humans, include HLA-DP, -DQ, and DR. The term “MHC restriction” refers to a characteristic of T cells that permits them to reorganize antigen only after it is processed and the resulting antigenic peptides are displayed in association with either a self class I or class II MHC molecule. Methods of identifying and comparing MHC are well known in the art and are described in Allen, M. et al. (1994) Human Immunol. 40:25-32; Santamaria, P. et al. (1993) Human Immunol. 37:39-50 and Hurley, C. K. et al. (1997) Tissue Antigens 50:401-415.

The term “antigen presenting cells (APC)” refers to a class of cells capable of presenting one or more antigens in the form of antigen-MHC complex recognizable by specific effector cells of the immune system, and thereby inducing an effective cellular immune response against the antigen or antigens being presented. While many types of cells may be capable of presenting antigens on their cell surface for T-cell recognition, only professional APCs have the capacity to present antigens in an efficient amount and further to activate T-cells for cytotoxic T-lymphocyte (CTL) response. APCs can be intact whole cells such as macrophages, B-cells and dendritic cells; or other molecules, naturally occurring or synthetic, such as purified MHC class I molecules complexed to beta2-microglobulin.

The term “dendritic cells (DC)” refers to a diverse population of morphologically similar cell types found in a variety of lymphoid and non-lymphoid tissues (Steinman (1991) Ann. Rev. Immunol. 9:271-296). Dendritic cells constitute the most potent and preferred APCs in the organism. At least a subset, if not all, dendritic cells are derived from bone marrow progenitor cells, circulate in small numbers in the peripheral blood and appear either as immature Langerhans' cells or terminally differentiated mature cells. While the dendritic cells can be differentiated from monocytes, they possess distinct phenotypes. For example, a particular differentiating marker, CD14 antigen, is either absent or present at low levels in dendritic cells, but is possessed by monocytes. Also, dendritic cells are not phagocytic, whereas the monocytes are strongly phagocytosing cells. It has been shown that DCs provide all the signals necessary for T cell activation and proliferation.

“Co-stimulatory molecules” are involved in the interaction between receptor-ligand pairs expressed on the surface of antigen presenting cells and T cells. Research accumulated over the past several years has demonstrated convincingly that resting T cells require at least two signals for induction of cytokine gene expression and proliferation (Schwartz R. H. (1990) Science 248:1349-1356 and Jenkins M. K. (1992) Immunol. Today 13:69-73). One signal, the one that confers specificity, can be produced by interaction of the TCR/CD3 complex with an appropriate MHC/peptide complex. The second signal is not antigen specific and is termed the “co-stimulatory” signal. This signal was originally defined as an activity provided by bone-marrow-derived accessory cells such as macrophages and dendritic cells, the so called “professional” APCs Several molecules have been shown to enhance co-stimulatory activity. These are heat stable antigen (HSA) (Liu Y. et al. (1992) J. Exp. Med. 175:437-445); chondroitin sulfate-modified MHC invariant chain (ICS) (Naujokas M. F. et al. (1993) Cell 74:257-268); intracellular adhesion molecule 1 (ICAM-1) (Van Seventer G. A. (1990) J. Immunol. 144:4579-4586); and B7-1 and B7-2/B70 (Schwartz R. H. (1992) Cell 71:1065-1068). Co-stimulatory molecules are commercially available from a variety of sources, including, for example, Beckmnan Coulter. It is intended, although not always explicitly stated, that molecules having similar biological activity as wild-type or purified co-stimulatory molecules (e.g., recombinantly produced or muteins thereof) are intended to be used within the spirit and scope of the invention.

As used herein, the term “cytokine” refers to any one of the numerous factors that exert a variety of effects on cells, for example, inducing growth or proliferation. Non-limiting examples of cytokines which may be used alone or in combination in the practice of the present invention include, interleukin-2 (IL-2), stem cell factor (SCF), interleukin 3 (IL-3), interleukin 6 (IL-6), interleukin 12 (IL-12), G-CSF, granulocyte macrophage-colony stimulating factor (GM-CSF), interleukin-1 alpha (IL-1), interleukin-11 (IL-11), MIP-1, leukemia inhibitory factor (LIF), c-kit ligand, thrombopoietin (TPO) and flt3 ligand. The present invention also includes culture conditions in which one or more cytokine is specifically excluded from the medium. Cytokines are commercially available from several vendors such as, for example, Genentech (South San Francisco, Calif.), Amgen (Thousand Oaks, Calif.), R&D Systems (Minneapolis, Minn.) and Immunex (Seattle, Wash.). It is intended, although not always explicitly stated, that molecules having similar biological activity as wild-type or purified cytokines (e g, recombinantly produced or muteins thereof) are intended to be used within the spirit and scope of the invention.

The term “culturing” refers to the in vitro propagation of cells or organisms on or in media of various kinds. It is understood that the descendants of a cell grown in culture may not be completely identical (i.e., morphologically, genetically, or phenotypically) to the parent cell. By “expanded” is meant any proliferation or division of cells.

A “composition” is intended to mean a combination of active agent and another compound or composition, inert (for example, a detectable agent or label) or active, such as an adjuvant.

A “pharmaceutical composition” is intended to include the combination of an active agent with a carrier, inert or active, making the composition suitable for diagnostic or therapeutic use in vitro, in vivo or ex vivo.

An “effective amount” is an amount sufficient to effect beneficial or desired results. An effective amount can be administered in one or more administrations, applications or dosages.

The terms “cancer,” “neoplasm,” and “tumor,” used interchangeably and in either the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g. by such procedures as CAT scan, magnetic resonance imaging (MRI), X-ray, ultrasound or palpation. Biochemical or immunologic findings alone may be insufficient to meet this definition. Tumor cells often express antigens which are tumor specific. The term “tumor associated antigen” or “TAA” refers to an antigen that is associated with or specific to a tumor.

As used herein, “solid phase support” is not limited to a specific type of support. Rather a large number of supports are available and are known to one of ordinary skill in the art. Solid phase supports include silica gels, resins, derivatized plastic films, glass beads, cotton, plastic beads, alumina gels. A suitable solid phase support may be selected on the basis of desired end use and suitability for various synthetic protocols. For example, for peptide synthesis, solid phase support may refer to resins such as polystyrene (e.g., PAM-resin obtained from Bachem Inc. Peninsula Laboratories, etc.), POLYHIPE^{\dot{O}} resin (obtained from Aminotech, Canada), polyamide resin (obtained from Peninsula Laboratories), polystyrene resin grafted with polyethylene glycol (TentaGel^{\dot{a}}, Rapp Polymere, Tubingen, Germany) or polydimethylacrylamide resin (obtained from Milligen/Biosearch, California). In a preferred embodiment for peptide synthesis, solid phase support refers to polydimethylacrylamide resin.

A “transgenic animal” refers to a genetically engineered animal or offspring of genetically engineered animals. The transgenic animal may contain genetic material from at least one unrelated organism (such as from a bacteria, virus, plant, or other animal) or may contain a mutation which interferes with expression of a gene product.

The present invention provides enhanced anti-tumor vaccines in which a polypeptide or polypeptides encoding tumor antigens are linked to a polypeptide or polypeptides encoding immunostimulatory factors associated with APC functions. The co-administration of tumor antigens and APC-associated stimulatory factors will not only enable adequate antigen presentation to endogenous APCs but also enhance functions of the APCs for 1) presentation of co-stimulatory signals; 2) migration to T-cell rich sites; 3) secretion of T-cell growth factors; or 4) secretion of chemokines for recruitment of immune effector cells.

The immunostimulatory factors of this invention include any polypeptide factors that modulate immune responses mediated by APC and corresponding T cells. For example, co-stimulatory factors that are differentially expressed in APCs can be used directly to boost the APC functions in vivo. Co-stimulatory factors have been described above and include, but not limited to, heat stable antigen (HSA); chondroitin sulfate-modified MHC invariant chain (Ii-CS); intracellular adhesion molecule 1; and B7-1 and B7-2/B70. Also, the immunostimulatory factors of the invention can be gene regulatory factors that modulate the expression and activity of the above-described APC-associated co-stimulatory factors. In addition, ligands of APC-associated co-stimulatory factors can be used to create an autocrine loop whereby the genetically modified APC secrets a soluble ligand which is then available to bind cell surface receptors and activate the APC.

For the purpose of this invention, polypeptides and the polynucleotides encoding cell-specific antigens can be, in one embodiment, previously characterized tumor-associated antigens such as melanoma-associated antigen gp 100 (Kawakami et al. (1997) Intern. Rev Immunol. 14:173-192); MNC-1 (Henderson et al. (1996) Cancer Res. 56:3763); MART-1 (Kawakami et al. (1994) Proc. Natl. Acad. Sci. 91:3515; Ribas et al. (1997) Cancer Res. 57:2865); HER-2/neu (U.S. Pat. No. 5,550,214); MAGE (PCT/US92/04354); HPVI6 18E6 and E7 (Ressing et al. (1996) Cancer Res. 56(1):582; Restifo (1996) Current Opinion in Immunol. 8:658; Stem (1996) Adv. Cancer Res. 69:175; Tindle et al. (1995) Clin. Exp. Immunol. 101:265; van Driel et al. (1996) Annals of Medicine 28:471); CEA (U.S. Pat. No. 5,274,087); PSA (Lundwall, A. (1989) Biochem. Biophys. Research Communications 161:1151); prostate specific membrane antigen (PSMA) (Israeli et al. (1993) Cancer Research 53:227); tyrosinase (U.S. Pat. Nos. 5,530,096 and 4,898,814; Brichard et al. (1993) J. Exp. Med. 178:489); tyrosinase related proteins 1 or 2 (TRP-1 and TRP-2): NYESO-1 (Chen et al. (1997) Proc. Natl. Acad. Sci. U.S.A. 94:1914), or the GA733 antigen (U.S. Pat. No. 5,185,254).

Selection of Immunostimulatory Factors of the Invention

Embodiments of the present invention include immunostimulatory factors that are preferentially or differentially expressed in monocyte-derived dedritic cells. Many comparative gene expression analysis can be used to identify genes and mRNAs preferentially or differentially expressed in monocyte-derived dedritic cells as compared to other cells such as the monocyte precursor cells. One preferred method is the SAGE analysis—Serial Analysis of Gene Expression (Velculescu, et al. (1995) Science 270:484-487 and U.S. Pat. No. 5,695,937).

SAGE provides the tool by which the expressed genes and the expression level of the genes of a cell at any one point in the cell cycle and under various environmental stimuli are isolated, sequenced and cataloged. SAGE provides quantitative gene expression data without the prerequisite of a hybridization probe for each transcript. SAGE is based on two principles. First, a short sequence tag (9-11 base pairs) contains sufficient information to uniquely identify a transcript, provided that it is derived from a defined location within that transcript. Second, many transcript tags can be concatenated into a single molecule and then sequenced, revealing the identity of multiple tags simultaneously. The expression pattern of any population of transcripts can be quantitatively evaluated by determining the abundance of individual tags and identifying the gene corresponding to each tag. Velculescu. et al. (1995) supra at 484.

Isolation and Characterization of Macromolecules of the Invention

In one embodiment, the present invention provides isolation and characterization of costimulatory factors preferentially or differentially expressed in APCs such as monocyte-derived dendritic cells. SAGE analysis, as described above, can be used to identify a population of sequence tags corresponding to gene transcripts that are preferably or differentially expressed in dendritic cells but not in their monocyte precursor cells. In one aspect, the transcript or gene is previously identified but was heretofore unknown to be preferentially or differentially expressed in monocyte-derived dendritic cells. In another embodiment, the transcript or gene disclosed herein is “novel”, which means the tag or its respective complement does not comprise sequence of or correspond to a previously identified expressed sequence tag (EST) or characterized gene.

As one non-exclusive example, SAGE analysis has revealed that the chemokines PARC and TARC (Pulmonary and Activation-Regulated Chemokine, Hieshima, et al. (1997) J. Imm. 159(3):1140-1149 and Thymus and Activation-Regulated Chemokine, Imai, et al. (1996) J. Biol. Chem. 271(35):21514-21521) known for capability to recruit activated T cells, are differentially expressed by monocyte-derived immature dendritic cells, a fact that has not been appreciated prior to the present invention.

In a separate embodiment of the invention, the immunostimulatory factor as claimed can be a co-stimulatory factor that is differentially expressed in monocyte-derived DCs. The costimulatory factor used herein will include at least a portion of the protein sufficient to allow binding to its costimulatory ligand expressed on corresponding T cell surface.

According to an alternative embodiment of the invention, genes encoding transcription factors capable of upregulating the expression and activity of above-discussed costimulatory factors are used. The encoded transcription factors can be naturally occurring proteins involved in gene regulation pathways for the differentially expressed costimulatory factors in dendritic cells. Examples of transcription factors include: Nuclear Factor kappa B (NFκB) [Grohmann et al., (1998), Immunity 9(3) p. 315-323], X61498; the rel family of proteins notably relB [Wu et al., (1998) Immunity 9(6) p. 839-847]; CCAAT/enhancer binding protein [Yamanaka et al., (1998) Bioorganic and Medicinal Chemistry Letters 1 (1) p. 213-221] Y1 1525; inteferon-stimulated gene factor 3 (ISGF-3) [Schindler et al., 1992 P.N.A.S. USA 89(16) p. 7836-7839] M97935; STAT5 [Welte et al., (1997) European Journal of Immunology 27(10) p. 2737-2740], U43185; and NFAT-X [Masuda et al., 1995 Molecular and Cellular Biology 15(5) p. 2697-2706] U14510. Alternatively, the nucleotides encoding transcription factors can be engineered via recombinant DNA technology. When included in a vaccine of the present invention, these nucleotides are capable of producing transcription factors that will transactivate the expression of the endogenous genes.

Thus, according to one embodiment of the invention, the immunostimulatory factor as claimed can be a transcription factor regulating the gene expression of a co-stimulatory factor differentially expressed in monocyte-derived DCs.

Differential gene expression analysis would also be expected to reveal genes encoding cell surface proteins that are preferentially expressed by either immature or mature dendritic cells as compared to monocyte precursors. Examples of such dendritic cell surface proteins that would be targets for activating ligands or engineered binding molecules (such as antibodies) include: IFN alpha/beta receptor X89814; IL-13 receptor Y09328; CD27 ligand L08096; CDlb M28826; CD151 D29963; CD53 M60871; LFA-1 M15395; and WSX1 cytokine receptor AF053004. These cell surface molecules may play a pivotal role in the function of dendritic cells by acting as co-stimulatory signals or modulators of DC function or migration. Naturally occurring ligands specific for these cell surface molecules or recombinant proteins (such as an antibody) generated to have specificity for these cell surface molecules might be expected to interact with the cell surface protein to stimulate the function of the dendritic cells or foster the maintenance of an activated state or stimulate the migration of dendritic cells to sites rich in T cells. Thus, the present invention relates to vaccines in which a gene or genes encoding tumor antigen or antigens is linked to a gene or genes encoding secreted proteins that have the capacity to bind to and modulate the activity of cell surface proteins identified as being differentially expressed in either mature or immature dendritic cells by comparative gene expression analysis (such as SAGE). In this manner, a novel autocrine loop is established whereby a genetically modified APC produces a ligand that is secreted from the APC where it can bind to cell surface receptors on that cell and stimulate the genetically modified dendritic cell.

In accordance with the present invention, the polynucleotide sequence encoding a tumor antigens and the polynucleotide sequence encoding an immunostimulatory factor can be constructed as separate molecules in the vaccine composition of the invention. Alternatively, the two nucleotide sequences can be covalently linked to form a single polynucleotide construct using standard recombinant DNA technology or chemical synthesis method. A recombinant DNA construct can be designed to have the linked sequences under the control of one transcriptional control region that can mediate the expression of both the tumor antigen and the immunostimulatory factor in a vaccine composition.

The invention also encompasses polynucleotides which differ from that of the polynucleotides described above, but encode substantially the same amino acid sequences. These altered, but phenotypically equivalent polynucleotides are referred to as “functionally equivalent nucleic acids.” As used herein, “functionally equivalent nucleic acids” encompass nucleic acids characterized by slight and non-consequential sequence variations that will function in substantially the same manner to produce the same protein product(s) as the nucleic acids disclosed herein (e.g. by virtue of the degeneracy of the genetic codes), or that have conservative amino acid variations. For example, conservative variations include substitution of a non-polar residue with another non-polar residue, or substitution of a charged residue with a similarly charged residue. These sequence variations include those recognized by artisans in the art as those that do not substantially alter the tertiary structure of the encoded protein.

The polynucleotides of the invention can comprise additional sequences, such as additional coding sequences within the same transcription unit, controlling elements such as promoters, ribosome binding sites, and polyadenylation sites, additional transcription units under control of the same or a different promoter, sequences that permit cloning, expression, and transformation of a host cell, and any such construct as may be desirable to provide embodiments of this invention.

Indeed, this invention also provides a promoter sequence derived from cell's genome, wherein the promoter sequence corresponds to the regulatory region of a gene that is differentially transcribed in the cell as compared to a control cell. The promoters are identified and characterized by: 1) probing a cDNA library with a probe corresponding to the SAGE tag sequence or generating a portion of the desired cDNA by conducting anchored PCR using primers based on the SAGE tag sequence. Examples of cell types wherein differential expression of a gene is related to promoter function include using the partial cDNA product obtained in step one above as a probe cloning the extreme 5′ end of the cDNA, and also by using the 5′ end of the cDNA as a probe, cloning from a genomic library the promoter of the gene that encodes the cDNA. These promoters are identified using the methods described below in combination with standard molecular techniques. Functionally equivalent sequences, as defined above, are further provided by this invention.

In one aspect, the promoter is a sequence derived from an APC genome, wherein the promoter region corresponds to the regulatory region of a gene that is differentially transcribed in the APC. In a further aspect, the APC is a TNF-α treated dendritic cell. In a yet further aspect, the APC is an immature dendritic cell. In a still further aspect, the expression of genes from these two cell sources are compared to genes expressed in monocytes from which the dendritic cell populations were derived.

The promoters identified above can be operatively linked to a foreign polynucleotide to compel differential transcription of the foreign polynucleotide in the cell from which the promoter was derived. A foreign polynucleotide is intended to include any sequence which encodes in whole or in part a polypeptide or protein. It also includes sequences encoding ribozymes and antisense molecules.

Foreign polynucleotides also include therapeutic genes that encode dominant inhibitory oligonucleotides and peptides as well as genes that encode regulatory proteins and oligonucleotides. Generally, gene therapy will involve the transfer of a single therapeutic gene although more than one gene may be necessary for the treatment of particular diseases. In one embodiment, the therapeutic gene is a dominant inhibiting mutant of the wild-type immunosuppressive agent. Alternatively, the therapeutic gene could be a wild-type copy of a defective gene or a functional homolog.

In one aspect, a tag identified in the Table corresponds to or comprises a polynucleotide that encodes a polypeptide or protein that is biologically active as an antigen, e.g., a native antigen, an altered antigen, a self-antigen or a tumor-associated antigen. Antigens are identified by noting the overexpression or cell-specific expression of a tag identified herein. Using the methods described below, the gene comprising or corresponding to the tag is identified, cloned and inserted into an APC. The tag corresponds to an antigen if a CTL response is raised under appropriate experimental conditions. The peptide is confirmed immunogeneic if an appropriate immune response is elecited.

The invention also encompasses co-administration of an immunostimulatory factor and a foreign polynucleotide, both under the control of promoters. In one embodiment, the promoter is an APC specific promoter. In alternative embodiment, the promoters are specific to tissue identified in the Table. The immunostimulatory factors of this invention include any polypeptide factors that modulate immune responses mediated by APC and corresponding T cells. For example, co-stimulatory factors that are differentially expressed in APCs can be used directly to boost the APC functions in vivo. Co-stimulatory factors have been described above.

The polynucleotides of the invention can be introduced and expressed in a suitable host cell for generating a cell-based vaccine. These methods are described in more detail below.

The polynucleotides and seqeunces identifed above can be conjugated to a detectable market, e.g., an enzymatic label or a radioisotope for detection of nucleic acid and/or expression of the gene in a cell. A wide variety of appropriate detectable markers are known in the art, including fluorescent, radioactive, enzymatic or other ligands, such as avidinibiotin, which are capable of giving a detectable signal. In preferred embodiments, one will likely desire to employ a fluorescent label or an enzyme tag, such as urease, alkaline phosphatase or peroxidase, instead of radioactive or other environmental undesirable reagents. In the case of enzyme tags, colorimetric indicator substrates are known which can be employed to provide a means visible to the human eye or spectrophotometrically, to identify specific hybridization with complementary nucleic acid-containing samples.

The polynucleotides and sequences embodied in this invention can be obtained using chemical synthesis, recombinant cloning methods, PCR, or any combination thereof. Methods of chemical polynucleotide synthesis are well known in the art and need not be described in detail herein. One of skill in the art can use the sequence data provided herein to obtain a desired polynucleotide by employing a DNA synthesizer or ordering from a commercial service.

The polynucleotides and sequences of this invention can be inserted into a suitable vector, and the vector in turn can be introduced into a suitable host cell for replication and/or amplification. Polynucleotides can be introduced into host cells by any means known in the art. Cells are transformed by introducing an exogenous polynucleotide by direct uptake, endocytosis, transfection, f-mating or electroporation. Once introduced, the exogenous polynucleotide can be maintained within the cell as a non-integrated vector (such as a plasmid) or integrated into the host cell genome. Amplified DNA can be isolated from the host cell by standard methods. See, e.g., Sambrook et al. (1989) supra, RNA can also be obtained from transformed host cell, or it can be obtained directly from the DNA by using a DNA-dependent RNA polymerase.

The present invention further encompasses a variety of gene delivery vehicles comprising the polynucleotide of the present invention. Gene delivery vehicles include both viral and non-viral vectors such as naked plasmid DNA or DNA/liposome complexes. Vectors are generally categorized into cloning and expression vectors. Cloning vectors are useful for obtaining replicate copies of the polynucleotides they contain, or as a means of storing the polynucleotides in a depository for future recovery. Expression vectors (and host cells containing these expression vectors) can be used to obtain polypeptides produced from the polynucleotides they contain. Suitable cloning and expression vectors include any known in the art, e.g., those for use in bacterial, mammalian, yeast and insect expression systems. The polypeptides produced in the various expression systems are also within the scope of the invention and are described above.

When the vectors are used for gene therapy in vivo or ex vivo, a pharmaceutically acceptable vector is preferred, such as a replication-incompetent retroviral or adenoviral vector. Pharmaceutically acceptable vectors containing the nucleic acids of this invention can be further modified for transient or stable expression of the inserted polynucleotide. As used herein, the term “pharmaceutically acceptable vector” includes, but is not limited to, a vector or delivery vehicle having the ability to selectively target and introduce the nucleic acid into dividing cells. An example of such a vector is a “replication-incompetent” vector defined by its inability to produce viral proteins, precluding spread of the vector in the infected host cell. An example of a replication-incompetent retroviral vector is LNL6 (Miller A. D. et al. (1989) BioTechniques 7:980-990). The methodology of using replication-incompetent retroviruses for retroviral-mediated gene transfer of gene markers is well established (Correll et al. (1989) Proc. Natl. Acad. Sci. USA 86:8912; Bordignon (1989) Proc. Natl. Acad. Sci. USA 86:8912-52; Culver K. (1991) Proc. Natl. Acad. Sci. USA 88:3155; and Rill, D. R. (1991) Blood 79(10):2694. Clinical investigations have shown that there are few or no adverse effects associated with the viral vectors, see Anderson (1992) Science 256:808-13.

Compositions containing the polynucleotides and sequences of this invention, in isolated form or contained within a vector or host cell are further provided herein. When these compositions are to be used pharmaceutically, they are combined with a pharmaceutically acceptable carrier.

A vector of this invention can contain one or more polynucleotides comprising a sequence shown in the Table or its complement. It can also contain polynucleotide sequences encoding other polypeptides that enhance, facilitate, or modulate the desired result, such as fusion components that facilitate protein purification, and sequences that increase immunogenicity of the resultant protein or polypeptide.

Also embodied in the present invention are host cells transformed with the vectors as described above. Both prokaryotic and eukaryotic host cells may be used. Prokaryotic hosts include bacterial cells, for example E. coli and Mycobacteria. Among eukaryotic hosts are yeast, insect, avian, plant and mammalian cells. Host systems are known in the art and need not be described in detail herein. Examples of mammalian host cells include but not limited to COS, HeLa, and CHO cells, and APCs, e.g., dendritic cells.

The host cells of this invention can be used, inter alia, as repositories of polynucleotides differentially expressed in a cell or as vehicles for production of the polynucleotides and the encoded polypeptides.

Polypeptides of the Invention

This invention provides a population of proteins or polypeptides expressed from the population of polynucleotides of this invention, which is intended to include wild-type and recombinantly produced polypeptides and proteins from prokaryotic and eukaryotic host cells, as well as muteins, analogs, fusions and fragments thereof. In some embodiments, the term also includes antibodies and anti-idiotypic antibodies.

It is understood that equivalents or variants of the wild-type polypeptide or protein also are within the scope of this invention. An “equivalent” varies from the wild-type sequence encoded by the polynucleotides of the invention by any combination of additions, deletions, or substitutions while preserving at least one functional property of the fragment relevant to the context in which it is being used. For instance, an equivalent of a polypeptide of the invention may have the ability to elicit an immune response with a similar antigen specificity as that elicited by the wild-type polypeptide. As is apparent to one skilled in the art. the equivalent may also be associated with, or conjugated with, other substances or agents to facilitate, enhance, or modulate its function.

The invention includes modified polypeptides containing conservative or non-conservative substitutions that do not significantly affect their properties, such as the immunogenicity of the peptides or their tertiary structures. Modification of polypeptides is routine practice in the art. Amino acid residues which can be conservatively substituted for one another include but are not limited to: glycine/alanine; valine/isoleucine/leucine; asparasine/glutamine; aspartic acid/glutamic acid; serine/threonine; lysine/arginine: and phenylalanine/tyrosine. These polypeptides also include glycosylated and nonglycosylated polypeptides, as well as polypeptides with other post-translational modifications, such as, for example, glycosylation with different sugars, acetylation, and phosphorylation.

The polypeptides of the invention can also be conjugated to a chemically functional moiety. Typically, the moiety is a label capable of producing a detectable signal. These conjugated polypeptides are useful, for example, in detection systems such as imaging of breast tumor. Such labels are known in the art and include, but are not limited to, radioisotopes, enzymes, fluorescent compounds, chemiluminescent compounds, bioluminescent compounds substrate cofactors and inhibitors. See, for examples of patents teaching the use of such labels, U.S. Pat. Nos. 3,817,837; 3,850,752; 3,939,350; 3,996,345; 4,277,437; 4,275,149; and 4,366,241. The moieties can be covalently linked to the polypeptides, recombinantly linked, or conjugated to the polypeptides through a secondary reagent, such as a second antibody, protein A, or a biotin-avidin complex.

Other functional moieties include agents that enhance immunological reactivity, agents that facilitate coupling to a solid support, vaccine carriers, bioresponse modifiers, paramagnetic labels and drugs. Agents that enhance immunological reactivity include, but are not limited to, bacterial superantigens. Agents that facilitate coupling to a solid support include, but are not limited to, biotin or avidin. Immunogen carriers include, but are not limited to, any physiologically acceptable buffers.

The invention also encompasses fusion proteins comprising polypeptides encoded by the polynucleotides disclosed herein and fragments thereof. Such fusion may be between two or more polypeptides of the invention and a related or unrelated polypeptide. Useful fusion partners include sequences that facilitate the intracellular localization of the polypeptide, or enhance immunological reactivity or the coupling of the polypeptide to an immunoassay support or a vaccine carrier. For instance, the polypeptides can be fused with a bioresponse modifier. Examples of bioresponse modifiers include, but are not limited to, fluorescent proteins such as green fluorescent protein (GFP), cytokines or lymphokines such as interleukin-2 (IL-2), interleukin 4 (IL-4), GM-CSF, and α-interferon. Another useful fusion sequence is one that facilitates purification. Examples of such sequences are known in the art and include those encoding epitopes such as Myc, HA (derived from influenza virus hemagglutinin), His-6, or FLAG. Other fusion sequences that facilitate purification are derived from proteins such as glutathione S-transferase (GST), maltose-binding protein (MBP), or the Fc portion of immunoglobulin. For immunological purposes, tandemly repeated polypeptide segments may be used as antigens, thereby producing highly immunogenic proteins.

The proteins of this invention also can be combined with various liquid phase carriers, such as sterile or aqueous solutions, pharmaceutically acceptable carriers, suspensions and emulsions. Examples of non-aqueous solvents include propyl ethylene glycol, polyethylene glycol and vegetable oils. When used to prepare antibodies, the carriers also can include an adjuvant that is useful to non-specifically augment a specific immune response. A skilled artisan can easily determine whether an adjuvant is required and select one. However, for the purpose of illustration only, suitable adjuvants include, but are not limited to Freund's Complete and Incomplete, mineral salts and polynucleotides.

The proteins and polypeptides of this invention are obtainable by a number of processes well known to those of skill in the art, which include purification, chemical synthesis and recombinant methods. Full-length proteins can be purified from a cell derived from non-metastatic or metastatic breast tumor tissue or tissue lysate by methods such as immunoprecipitation with antibody, and standard techniques such as gel filtration, ion-exchange, reversed-phase, and affinity chromatography using a fusion protein as shown herein. For such methodology, see for example Deutscher et al. (1999) GUIDE TO PROTEIN PURIFICATION: METHODS IN ENZYMOLOGY (Vol. 182, Academic Press). Accordingly, this invention also provides the processes for obtaining these proteins and polypeptides as well as the products obtainable and obtained by these processes.

The proteins and polypeptides also can be obtained by chemical synthesis using a commercially available automated peptide synthesizer such as those manufactured by Perkin Elmer/Applied Biosystems, Inc., Model 430A or 431A, Foster City, Calif., USA. The synthesized protein or polypeptide can be precipitated and farther purified, for example by high performance liquid chromatography (HPLC). Accordingly, this invention also provides a process for chemically synthesizing the proteins of this invention by providing the sequence of the protein and reagents, such as amino acids and enzymes and linking together the amino acids in the proper orientation and linear sequence.

Alternatively, the proteins and polypeptides can be obtained by well-known recombinant methods as described, for example, in Sambrook et al. (1989), supra, using the host cell and vector systems described above.

Antibodies

Also provided by this invention is a population of antibodies capable of specifically binding to the proteins or polypeptides as described above. The antibodies of the present invention encompass polyclonal antibodies and monoclonal antibodies. They include but are not limited to mouse, rat, and rabbit or human antibodies. This invention also encompasses functionally equivalent antibodies and fragments thereof. As used herein with respect to the exemplified antibodies, the phrase “functional equivalent” means an antibody or fragment thereof, or any molecule having the antigen binding site (or epitope) of the antibody that cross-blocks an exemplified antibody when used in an immunoassay such as immunoblotting or immunoprecipitation Antibody fragments include the Fab, Fab′, F(ab′)₂, and Fv regions, or derivatives or combinations thereof Fab, Fab′, and F(ab′), regions of an immunoglobulin may be generated by enzymatic digestion of the monoclonal antibodies using techniques well known to those skilled in the art. Fab fragments may be generated by digesting the monoclor al antibody with papain and contacting the digest with a reducing agent to reductively cleave disulfide bonds. Fab′ fragments may be obtained by digesting the antibody with pepsin and reductive cleavage of the fragment so produce with a reducing agent. In the absence of reductive cleavage, enzymatic digestion of the monoclonal with pepsin produces F(ab′)₂ fragments.

It will further be appreciated that encompassed within the definition of antibody fragment is single chain antibody that can be generated as described in U.S. Pat. No. 4,704,692, as well as chimeric antibodies and humanized antibodies (Oi et al. (1986) BioTechniques 4(3):214). Chimeric antibodies are those in which the various domains of the antibodies' heavy and light chains are coded for by DNA from more than one species.

As used herein with regard to the monoclonal antibody, the “hybridoma cell line” is intended to include all derivatives, progeny cells of the parent hybridoma that produce the monoclonal antibodies specific for the polypeptides of the present invention, regardless of generation of karyotypic identity.

Laboratory methods for producing polyclonal antibodies and monoclonal antibodies, as well as deducing their corresponding nucleic acid sequences, are known in the art, see Harlow and Lane (1988) supra and Sambrook et al. (1989) supra. For production of polyclonal antibodies, an appropriate host animal is selected, typically a mouse or rabbit. The substantially purified antigen, whether the whole transmembrane domain, a fragment thereof, or a polypeptide corresponding to a segment of or the entire specific loop region within the transmembrane domain, coupled or fused to another polypeptide, is presented to the immune system of the host by methods appropriate for the host. The antigen is introduced commonly by injection into the host footpads, via intramuscular, intraperitoneal, or intradermal routes. Peptide fragments suitable for raising antibodies may be prepared by chemical synthesis, and are commonly coupled to a carrier molecule (e.g., keyhole limpet hemocyanin) and injected into a host over a period of time suitable for the production of antibodies. Alternatively, the antigen can be generated recombinantly as a fusion protein. Examples of components for these fusion proteins include, but are not limited to myc, HA, is FLAG, His-6, glutathione S-transferease, maltose binding protein or the Fc portion of immunoglobulin.

The monoclonal antibodies of this invention refer to antibody compositions having a homogeneous antibody population. It is not intended to be limited as regards to the source of the antibody or the manner in which it is made. Generally, monoclonal antibodies are biologically produced by introducing protein or a fragment thereof into a suitable host, e.g., a mouse. After the appropriate period of time, the spleens of such animal is excised and individual spleen cells fused, typically, to immortalized myeloma cells under appropriate selection conditions. Thereafter the cells are clonally separated and the supernatants of each clone are tested for their production of an appropriate antibody specific for the desired region of the antigen using methods well known in the art.

The isolation of other hybridomas secreting monoclonal antibodies with the specificity of the monoclonal antibodies of the invention can also be accomplished by one of ordinary skill in the art by producing anti-idiotypic antibodies (Herlyn et al. (1986) Science 232:100). An anti-idiotypic antibody is an antibody which recognizes unique determinants present on the monoclonal antibody produced by the hybridoma of interest.

Idiotypic identity between monoclonal antibodies of two hybridomas demonstrates that the two monoclonal antibodies are the same with respect to their recognition of the same epitopic determinant. Thus, by using antibodies to the epitopic determinants on a monoclonal antibody it is possible to identify other hybridomas expressing monoclonal antibodies of the same epitopic specificity.

It is also possible to use the anti idiotype technology to produce monoclonal antibodies which mimic an epitope. For example, an anti-idiotypic monoclonal antibody made to a first monoclonal antibody will have a binding domain in the hypervariable region which is the mirror image of the epitope bound by the first monoclonal antibody. Thus, in this instance, the anti-idiotypic monoclonal antibody could be used for immunization for production of these antibodies.

Other suitable techniques of antibody production include, but are not limited to, in vitro exposure of lymphocytes to the antigenic polypeptides or selection of libraries of antibodies in phage or similar vectors See Huse et al. (1989) Science 246:1275-1281. Genetically engineered variants of the antibody can be produced by obtaining a polynucleotide encoding the antibody, and applying the general methods of molecular biology to introduce mutations and translate the variant. The above described antibody “derivatives” are further provided herein.

Sera harvested from the immunized animals provide a source of polyclonal antibodies. Detailed procedures for purifying specific antibody activity from a source material are known within the art. Undesired activity cross-reacting with other antigens, if present, can be removed, for example, by running the preparation over adsorbants made of those antigens attached to a solid phase and eluting or releasing the desired antibodies off the antigens. If desired, the specific antibody activity can be further purified by such techniques as protein A chromatography, ammonium sulfate precipitation, ion exchange chromatography, high-performance liquid chromatography and immunoaffinity chromatography on a column of the immunizing polypeptide coupled to a solid support.

The specificity of an antibody refers to the ability of the antibody to distinguish polypeptides comprising the immunizing epitope from other polypeptides. An ordinary skill in the art can readily determine without undue experimentation whether an antibody shares the same specificity as a antibody of this invention by determining whether the antibody being tested prevents an antibody of this invention from binding the polypeptide(s) with which the antibody is normally reactive. If the antibody being tested competes with the antibody of the invention as shown by a decrease in binding by the antibody of this invention, then it is likely that the two antibodies bind to the same or a closely related epitope. Alternatively, one can pre-incubate the antibody of this invention with the polypeptide(s) with which it is normally reactive, and is determine if the antibody being tested is inhibited in its ability to bind the antigen. If the antibody being tested is inhibited, then, in all likelihood, it has the same, or a closely related, epitopic specificity as the antibody of this invention.

The antibodies of the invention can be bound to many different carriers. Thus, this invention also provides compositions containing antibodies and a carrier. Carriers can be active and/or inert. Examples of well-known carriers include polypropylene, polystyrene, polyethylene, dextran, nylon, amylases, glass, natural and modified celluloses, polyacrylamides, agaroses and magnetite. The nature of the carrier can be either soluble or insoluble for purposes of the invention. Those skilled in the art will know of other suitable carriers for binding antibodies, or will be able to ascertain such, using routine experimentation.

The antibodies of this invention can also be conjugated to a detectable agent or a hapten. The complex is useful to detect the polypeptide(s) (or polypeptide fragments) to which the antibody specifically binds in a sample, using standard immunochemical techniques such as immunohistochemistry as described by Harlow and Lane (1988) supra. There are many different labels and methods of labeling known to those of ordinary skill in the art. Examples of the types of labels which can be used in the present invention include radioisotopes, enzymes, colloidal metals, fluorescent compounds, bioluminescent compounds, and chemiluminescent compounds. Those of ordinary skill in the art will know of other suitable labels for binding to the antibody, or will be able to ascertain such, using routine experimentation. Furthermore, the binding of these labels to the antibody of the invention can be done using standard techniques common to those of ordinary skill in the art.

Another technique which may also result in greater sensitivity consists of coupling the antibodies to low molecular weight haptens. These haptens can then be specifically detected by means of a second reaction. For example, it is common to use such haptens as biotin, which reacts avidin, or dinitropherryl, pyridoxal, and fluorescein, which can react with specific anti-hapten antibodies. See Harlow and Lane (1988) supra.

Compositions containing the antibodies, fragments thereof or cell lines which produce the antibodies, are encompassed by this invention. When these compositions are to be used pharmaceutically, they are combined with a pharmaceutically acceptable carrier.

Uses of Polynucleotides, Polypeptides and Antibodies of the Invention

The polynucleotides, polypeptides and antibodies embodied in this invention provide specific reagents that can be used in standard diagnostic procedures. Accordingly, one embodiment of the present invention is a method of characterizing a cell of monocyte lineage by detecting differential expression of a polynucleotide comprising any one of the sequences shown in the Table or any one of the disclosed populations, or the encoded polypeptides.

In assaying for an alteration in mRNA level, nucleic acid contained in the aforementioned a sample suspected of containing a dendritic cell is first extracted according to standard methods in the art. For instance, mRNA can be isolated using various lytic enzymes or chemical solutions according to the procedures set forth in Sambrook et al. (1989), supra or extracted by nucleic-acid-binding resins following the accompanying instructions provided by manufactures. The mRNA contained in the extracted nucleic acid sample is then detected by hybridization (e.g. Northern blot analysis) and/or amplification procedures according to methods widely known in the art or based on the methods exemplified herein.

Nucleic acid molecules having at least 10 nucleotides and exhibiting sequence complementarity or homology to the polynucleotides described herein find utility as hybridization probes. It is known in the art that a “perfectly matched” probe is not needed for a specific hybridization. Minor changes in probe sequence achieved by substitution, deletion or insertion of a small number of bases do not affect the hybridization specificity. In general, as much as 20% base-pair mismatch (when optimally aligned) can be tolerated. Preferably, a probe useful for detecting the aforementioned mRNA that is differentially expressed in the cell type for which one is probing. These tags are identified in Table 1, below. More preferably, the probe is at least 80%, or 85% identical to the corresponding gene sequence after alignment of the homologous region; even more preferably, it exhibits 90% identity.

These probes can be used in hybridization reaction (e.g. Southern and Northern blot analysis) to detect, prognose, diagnose or monitor the physiological states associated with the differential expression of these genes. The total size of fragment, as well as the size of the complementary stretches, will depend on the intended use or application of the particular nucleic acid segment. Smaller fragments derived from the known sequences will generally find use in hybridization embodiments, wherein the length of the complementary region may be varied, such as between about 10 and about 100 nucleotides, or even full length according to the complementary sequences one wishes to detect.

Nucleotide probes having complementary sequences over stretches greater than 10 nucleotides in length are generally preferred, so as to increase stability and selectivity of the hybrid, and thereby improving the specificity of particular hybrid molecules obtained. More preferably, one can design nucleic acid molecules having gene-complementary stretches of more than 50 nucleotides in length, or even longer where desired. Such fragments may be readily prepared by, for example, directly synthesizing the fragment by chemical means, by application of nucleic acidreproduction technology, such as the PCR™ technology with two priming oligonucleotides as described in U.S. Pat. No. 4,603,102 or by introducing selected sequences into recombinant vectors for recombinant production. A preferred probe is about 50-75 or more preferably, 50-100, nucleotides in length.

In certain embodiments, it will be advantageous to employ nucleic acid sequences of the present invention in combination with an appropriate means, such as a label, for detecting hybridization and therefore complementary sequences. A wide variety of appropriate indicator means are known in the art, including fluorescent, radioactive, enzymatic or other ligands, such as avidin/biotin, which are capable of giving a detectable signal. In preferred embodiments, one will likely desire to employ a fluorescent label or an enzyme tag, such as urease, alkaline phosphatase or peroxidase, instead of radioactive or other environmental undesirable reagents. In the case of enzyme tags, colorimetric indicator substrates are known which can be employed to provide a means visible to the human eye or spectrophotometrically, to identify specific hybridization with complementary nucleic acid-containing samples.

The nucleotide probes of the present invention can also be used as primers and detection of genes or gene transcripts that are differentially expressed in certain body tissues. A preferred primer is one comprising a sequence of shown in the Table or their respective complements. Additionally, a primer useful for detecting the aforementioned gene or transcript is at least about 80% identical to the homologous region of comparable size of the gene or transcript to be detected contained in the previously identified sequences. For the purpose of this invention, amplification means any method employing a primer-dependent polymerase capable of replicating a target sequence with reasonable fidelity. Amplification may be carried out by natural or recombinant DNA-polymerases such as T7 DNA polymerase, Klenow fragment of E.coli DNA polymerase, and reverse transcriptase.

A preferred amplification method is PCR. General procedures for PCR are taught in MacPherson et al., PCR: A PRACTICAL APPROACH, (IRL Press at Oxford University Press (1991)). However, PCR conditions used for each application reaction are empirically determined. A number of parameters influence the success of a reaction. Among them are annealing temperature and time, extension time, Mg²⁺ ATP concentration, pH, and the relative concentration of primers, templates, and deoxyribonucleotides.

After amplification, the resulting DNA fragments can be detected by agarose gel electrophoresis followed by visualization with ethidium bromide staining and ultraviolet illumination. A specific amplification of the gene or transcript of interest can be verified by demonstrating that the amplified DNA fragment has the predicted size, exhibits the predicated restriction digestion pattern, and/or hybridizes to the correct cloned DNA sequence.

The probes and tags of this invention also can be attached to a solid support for use in high throughput screening assays using methods known in the art. PCT WO 97/10365 and U.S. Pat. Nos. 5,405,783, 5,412,087 and 5,445,934, for example, disclose the construction of high density oligonucleotide chips which can contain one or more of the sequences disclosed herein. Based in the methods disclosed in U.S. Pat. Nos. 5,405,783, 5,412,087 and 5,445,934, the probes of this invention are synthesized on a derivatized glass surface. Photoprotected nucleoside phosphoramidites are coupled to the glass surface, selectively deprotected by photolysis through a photolithographic mask, and reacted with a second protected nucleoside phosphoramidite. The coupling/deprotection process is repeated until the desired probe is complete.

The expression level of a gene of interest is determined through exposure of a nucleic acid sample to the probe-modified chip. Extracted nucleic acid is labeled, for example, with a fluorescent tag, preferably during an amplification step. Hybridization of the labeled sample is performed at an appropriate stringency level. The degree of probe-nucleic acid hybridization is quantitatively measured using a detection device, such as a confocal microscope. See U.S. Pat Nos. 5,578,832 and 5,631,734. The obtained measurement is directly correlated with gene expression level.

More specifically, the probes and high density oligonucleotide probe arrays provide an effective means of monitoring expression of a multiplicity of genes. The expression monitoring methods of this invention may be used in a wide variety of circumstances including detection of disease, identification of differential gene expression between two samples, or screening for compositions that upregulate or downregulate the expression of particular genes.

In another preferred embodiment, the methods of this invention are used to monitor expression of the genes which specifically hybridize to the probes of this invention in response to defined stimuli, such as a drug.

In one embodiment, the hybridized nucleic acids are detected by detecting one or more labels attached to the sample nucleic acids. The labels may be incorporated by any of a number of means well known to those of skill in the art. However, in one aspect, the label is simultaneously incorporated during the amplification step in the preparation of the sample nucleic acid. Thus, for example, polymerase chain reaction (PCR) with labeled primers or labeled nucleotides will provide a labeled amplification product. In a separate embodiment, transcription amplification, as described above, using a labeled nucleotide (e.g. fluorescein-labeled UTP and/or CTP) incorporates a label in to the transcribed nucleic acids.

Alternatively, a label may be added directly to the original nucleic acid sample (e.g., mRNA, polyA, mRNA, cDNA, etc.) or to the amplification product after the amplification is completed. Means of attaching labels to nucleic acids are well known to those of skill in the art and include, for example nick translation or end-labeling (e.g. with a labeled RNA) by kinasing of the nucleic acid and subsequent attachment (ligation) of a nucleic acid linker joining the sample nucleic acid to a label (e.g., a fluorophore).

The nucleic acid sample also may be modified prior to hybridization to the high density probe array in order to reduce sample complexity thereby decreasing background signal and improving sensitivity of the measurement using the methods disclosed in WO 97/10365.

Results from the chip assay are typically analyzed using a computer software program. See, for example, EP 0717 113 A2 and WO 95/20681. The hybridization data are read into the program, which calculates the expression level of the targeted gene(s). This figure is compared against existing data sets of gene expression levels for various cell types.

Expression of the genes associated characteristic of dendritic cells as compared to monocytes can also be determined by examining the protein product of the polynucleotides of the present invention. Determining the protein level involves a) providing a biological sample containing polypeptides; and (b) measuring the amount of any immunospecific binding that occurs between an antibody reactive to the protein products of interest and a component in the sample, in which the amount of immunospecific binding indicates the level of the protein products.

A variety of techniques are available in the art for protein analysis. They include but are not limited to radioimmunoassays, ELISA (enzyme linked immunoradiometric assays), “sandwich” immunoassays, immunoradiometric assays, in situ immunoassays (using e.g., colloidal gold, enzyme or radioisotope labels), western blot analysis, immunoprecipitation assays, immunofluorescent assays, and SDS-PAGE. In addition, cell sorting analysis can be employed to detect cell surface antigens. Such analysis involves labeling target cells with antibodies coupled to a detectable agent, and then separating the labeled cells from the unlabeled ones in a cell sorter. A sophisticated cell separation method is fluorescence-activated cell sorting (FACS). Cells traveling in single file in a fine stream are passed through a laser beam, and the fluorescence of each cell bound by the fluorescently labeled antibodies is then measured.

Antibodies that specifically recognize and bind to the protein products of interest are required for conducting the aforementioned protein analyses. These antibodies may be purchased from commercial vendors or generated and screened using methods well known in the art. See Harlow and Lane (1988) supra, and Sambrook et al. (1989) supra.

There are various methods available in the art for quantifying mRNA or protein level from a cell sample and indeed, any method that can quantify these levels is encompassed by this invention. For example, determination of the mRNA level of the gene may involve, in one aspect, measuring the amount of mRNA in a mRNA sample isolated from the cell by hybridization or quantitative amplification using at least one oligonucleotide probe that is complementary to the mRNA. Determination of the aforementioned protein products requires measuring the amount of immunospecific binding that occurs between an antibody reactive to the product of interest. To detect and quantify the immunospecific binding, or signals generated during hybridization or amplification procedures, digital image analysis systems including but not limited to those that detect radioactivity of the probes or chemiluminescence can be employed.

The promoter sequences of this invention are useful for targeted expression of foreign polynucleotides. The promoters can be operatively linked to foreign polynucleotides and administered to patients alone or after transduction into host cells. Because the promoters have been selected for cell-specific expression, after incorporation into host cells, either in vivo or ex vivo, targeted expression of the inserted polynucleotide can be obtained.

In one embodiment, the promoters preferentially express operatively linked polynucleotides in APCs, e.g., tumor associated antigens. Genes coding for immunostimulatory molecules such as cytokines or co-stimulatory molecules can be linked to the promoter sequence and gene coding for the antigen. Administration of these polynucleotides to a subject, alone or transduced into host APCs, are useful to induce an immune response by educating immune effector cells in vivo.

Screening Assays

The present invention also provides a screen for various agents which modulate the expression of a polynucleotide associated the phenotype of a normal or pathological cell by first contacting a suitable cell with an effective amount of a potential agent, and then assaying for a change in the expression level of a polynucleotide selected from the group consisting of SEQ ID NOS: 1-207 or any one of the populations from which the population was derived. For example, one would assay for a change in the expression level of any one of the polynucleotides comprising or corresponding to the sequence shown in the table in a monocyte or dendritic cell. A change in the expression level is indicative of a candidate modulating agent. In certain aspects of the invention, an agent may result in phenotypic changes of the recipient cell as evidenced by an agent-induced cell apoptosis, a reduced rate of cell growth or cell motility. Altered gene expression can be detected by assaying for altered mRNA expression or protein expression using the probes, primers and antibodies as described herein.

To practice the method in vitro, cell cultures or tissue cultures previously identified as expressing one or more of the tags or polypeptides corresponding to the tags are first provided. The cell can be a cultured cell or a genetically modified cell in which a transcript from the Table, or their complements, or alternatively, transcripts which contain or correspond to a tag or its respective complement is expressed. The cells are cultured under conditions (temperature, growth or culture medium and gas (CO₂)) and for an appropriate amount of time to attain exponential proliferation without density dependent constraints. It also is desirable to maintain an additionalseparate cell culture; one which does not receive the agent being tested as a control.

As is apparent to one of skill in the art, suitable cells may be cultured in microtiter plates and several agents may be assayed at the same time by noting genotypic changes and/or phenotypic changes.

When the agent is a composition other than naked DNA or RNA, the agent may be directly added to the cell culture or added to culture medium for addition. As is apparent to those skilled in the art, an “effective” amount must be added which can be empirically determined. When the agent is a polynucleotide, it may be introduced directly into a cell by transfection or electroporation. Alternatively, it may be inserted into the cell using a gene delivery vehicle or other methods as described above.

For the purposes of this invention, an “agent” is intended to include, but not be limited to a biological or chemical compound such as a simple or complex organic or inorganic molecule, a peptide, a protein (e.g. antibody) or a polynucleotide (e.g. anti-sense). A vast array of compounds can be synthesized, for example polymers, such as polypeptides and polynucleotides, and synthetic organic compounds based on various core structures, and these are also included in the term “agent.” In addition, various natural sources can provide compounds for screening, such as plant or animal extracts, and the like. It should be understood, although not always explicitly stated that the agent is used alone or in combination with another agent, having the same or different biological activity as the agents identified by the inventive screen. The agents and methods also are intended to be combined with other therapies.

The assays also can be performed in a subject. When the subject is an animal such as a rat, mouse or simian, the method provides a convenient animal model system which can be used prior to clinical testing of an agent. In this system, a candidate agent is a potential drug if transcript expression is altered, i.e., upregulated (such as restoring tumor suppressor function), downregulated or eliminated as with drug resistant genes or oncogenes, or if symptoms associated or correlated to the presence of cells containing transcript expression are ameliorated, each as compared to untreated, animal having the pathological cells. It also can be useful to have a separate negative control group of cells or animals which are healthy and not treated, which provides a basis for comparison. After administration of the agent to subject, suitable cells or tissue samples are collected and assayed for altered gene expression.

These agents of this invention and the above noted compounds and their derivatives can be combined with a pharmaceutically acceptable carrier for the preparation of medicaments for use in the methods described herein.

The agents of the present invention can be administered to a cell or a subject by various delivery systems known in the art. Non-limiting examples include encapsulation in liposomes, microparticles, microcapsules, expression by recombinant cells, receptor-mediated endocytosis (see, e g., Wu and Wu (1987) J. Biol. Chem. 262:4429-4432), and construction of a therapeutic nucleic acid as part of a retroviral or other vector. Methods of delivery include but are not limited to transdermally, gene therapy, intra-arterial, intramuscular, intravenous, intranasal, and oral routes, and include sustained delivery systems. In a specific embodiment, it may be desirable to administer the pharmaceutical compositions of the invention locally to the area in need of treatment; this may be achieved by, for example, and not by way of limitation, local infusion during surgery, by injection, or by means of a catheter or targeted gene delivery of the sequence coding for the therapeutic.

Administration in vivo can be effected in one dose, continuously or intermittently throughout the course of treatment. Methods of determining the most effective means and dosage of administration are well known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents can be found below.

The agents and compositions of the present invention can be used in the manufacture of medicaments and for the treatment of humans and other animals by administration in accordance with conventional procedures, such as an active ingredient in pharmaceutical compositions.

The pharmaceutical compositions can be administered orally, intranasally, parenterally, transdermally or by inhalation therapy, and may take the form of tablets, lozenges, granules, capsules, pills, ampoules, suppositories or aerosol form. They may also take the form of gene therapy, suspensions, solutions and emulsions of the active ingredient in aqueous or nonaqueous diluents, syrups, granulates or powders. In addition to an agent of the present invention, the pharmaceutical compositions can also contain other pharmaceutically active compounds or a plurality of compounds of the invention.

It should be understood that in addition to the ingredients particularly mentioned above, the formulations of this invention may include other agents conventional in the art having regard to the type of formulation in question, for example, those suitable for oral administration may include such further agents as sweeteners, thickeners and flavoring agents. It also is intended that the agents, compositions and methods of this invention be combined with other suitable compositions and therapies.

Genomics Applications

This invention also provides a process for preparing a database for the analysis of a cell's expressed genes by storing in a digital storage medium information related to the sequences of the transcriptome. Using this method, a data processing system for standardized representation of the expressed genes of a cell is compiled. The data processing system is useful to analyze gene expression between two cells by first selecting a cell and then identifying and sequencing the transcriptome of the cell. This information is stored in a computer-readable storage medium as the transcriptome. The transcriptome is then compared with at least one sequence(s) of transcription fragments from a reference cell. The compared sequences are then analyzed. Uniquely expressed sequences and sequences differentially expressed between the reference cell and the selected cell can be identified by this method.

In other words, this invention provides a computer based method for screening the homology of an unknown DNA or mRNA sequence against one or more of transcribed or expressed genes of a preselected cell by first providing the complete set of expressed genes, i.e., the transcriptome, in computer readable form and homology screening the DNA or mRNA of the unknown sequence against transcriptome and determining whether the DNA sequence of the unknown contains similarities to any portion of the transcriptome listed in the computer readable form. In one embodiment, SEQ ID Nos. or polynucleotides corresponding to these sequences are the transcriptome against which test cells are compared Thus, the information provided herein also provides a means to compare the relative abundance of gene transcripts in different biological specimens by use of high-throughput sequence-specific analysis of individual RNAs or their corresponding cDNAs using a modification of the systems described in WO 95/2068, 96/23078 and 5,618,672.

The tags or transcripts also can be attached to a solid support for use in high throughput screening assays. PCT WO 97/10365, for example, discloses the construction of high density oligonucleotide chips. See also, U.S. Pat. Nos. 5,405,783, 5,412,087 and 5,445,934. Using this method, the probes are synthesized on a derivatized glass surface. Photoprotected nucleoside phosphoramidites are coupled to the glass surface, selectively deprotected by photolysis through a photolithographic mask, and reacted with a second protected nucleoside phosphoramidite. The coupling/deprotection process is repeated until the desired probe is complete.

The expression level of a gene is determined through exposure of a nucleic acid sample to the probe-modified chip. Extracted nucleic acid is labeled, for example, with a fluorescent tag, preferably during an amplification step. Hybridization of the labeled sample is performed at an appropriate stringency level. The degree of probe-nucleic acid hybridization is quantitatively measured using a detection device, such as a confocal microscope. See U.S. Pat. Nos. 5,578,832 and 5,631,734. The obtained measurement is directly correlated with gene expression level.

Results from the chip assay are typically analyzed using a computer software program. See, for example, EP 0717 113 A2 and WO 95/20681. The hybridization data is read into the program, which calculates the expression level of the targeted gene(s). This figure is compared against existing data sets of gene expression levels

for that cell type.

Additional utilities of the database include, but are not limited to analysis of the developmental state of a test cell, the influence of viral or bacterial infection, control of cell cycle, effect of a tumor suppressor gene or lack thereof, polymorphism within the cell type, apoptosis, and the effect of regulatory genes.

Non-Human Transgenic Animals

In another aspect, the novel polynucleotide sequences associated with a pathological state of a cell can be used to generate transgenic animal models. In recent years, geneticists have succeeded in creating transgenic animals, for example mice, by manipulating the genes of developing embryos and introducing foreign genes into these embryos. Once these genes have integrated into the genome of the recipient embryo, the resulting embryos or adult animals can be analyzed to determine the function of the gene. The mutant animals are produced to understand the function of known genes in vivo and to create animal models of human diseases. (see, e.g.,Chisaka et al. (1992) 355.5 1 6-520; Joyner et al. (1 992) in POSTIMPLANTATION DEVELOPMENT IN THE MOUSE (Chadwick and Marsh, eds., John Wiley & Sons, United Kingdom) pp:277-297; Donrn et al. (1992) Nature 359:211-215).

The following examples are intended to illustrate, but not limit, the invention.

Cloning Techniques

The following are several techniques available to the skilled artisan for identification and cloning of the polynucleotides corresponding to the tags having the sequences set forth in the Table.

1) RACE-PCR Technique

One method to isolate the gene or cDNA which codes for a polypeptide or protein involves the 5′-RACE-PCR technique. In this technique, the poly-A mRNA that contains the coding sequence of particular interest is first identified by hybridization to a sequence disclosed herein and then reverse transcribed with a 3′-primer comprising the sequence disclosed herein. The newly synthesized cDNA strand is then tagged with an anchor primer of a known sequence, which preferably contains a convenient cloning restriction site attached at the 5′ end. The tagged cDNA is then amplified with the 3′-primer (or a nested primer sharing sequence homology to the internal sequences of the coding region) and the 5′-anchor primer. The amplification may be conducted under conditions of various levels of stringency to optimize the amplification specificity. 5′RACE-PCR can be readily performed using commercial kits (available from, e.g., BRL 5 Life Technologies Inc., Clontech) according to the manufacturer's instructions.

2) Isolation of Partial cDNA (3′ Fragment) by 3′ Directed PCR Reaction

This procedure is a modification of the protocol described in Polyak et al. (1997) Nature 389:300. Briefly, the procedure uses SAGE tags in PCR reaction such that the resultant PCR product contains the SAGE tag of interest as well as additional cDNA, the length of which is defined by the position of the tag with respect to the 3′ end of the cDNA. The cDNA product derived from such a transcript driven PCR reaction can be used for many applications.

RNA from a source believed to express the cDNA corresponding to a given tag is first converted to double-stranded cDNA using any standard cDNA protocol. Similar conditions used to generate cDNA for SAGE library construction can be employed except that a modified oligo-dT primer is used to derive the first strand synthesis. For example, the oligonucleotide of composition 540 -Biotin-TCC GGC GCG CCG TTT TCC CAG TCA CGA₍₃₀₎-3′ (SEQ ID NO:208), contains a poly-T stretch at the 3′ end for hybridization and priming from poly-A tails, an M13 priming site for use in subsequent PCR steps, a 5′ Biotin label (B) for capture to strepavidin-coated magnetic beads, and an AscI restriction endonuclease site for releasing the cDNA from the streptavidin-coated magnetic beads. Theoretically, any sufficiently-sized DNA region capable of hybridizing to a PCR primer can be used as well as any other 8 base pair recognizing endonuclease.

cDNA constructed utilizing this or similar modified oligo-dT primer is then processed exactly as described in U.S. Pat. No. 5,695,937 up until adapter ligation where only one adapter is ligated to the cDNA pool. After adapter ligation, the cDNA is released from the streptavidin-coated magnetic beads and is then used as a template for cDNA amplification.

Various PCR protocols can be employed using PCR priming sites within the 3′ modified oligo-dT primer and the SAGE tag. The SAGE tag-derived PCR primer employed can be of varying length dictated by 5′ extension of the tag into the adaptor sequence. cDNA products are now available for a variety of applications.

This technique can be further modified by: (1) altering the length and/or content of the modified oligo-dT primer; (2) ligating adaptors other than that previously employed within the SAGE protocol; (3) performing PCR from template retained on the streptavidin-coated magnetic beads; and (4) priming first strand cDNA synthesis with non-oligo-dT based primers.

3) Isolation of cDNA using GeneTrapper or Modified GeneTrapper TECHNOLOGY

The reagents and manufacturer's instructions for this technology are commercially available from Life Technologies, Inc., Gaithersburg, Md. Briefly a complex population of single-stranded phagemid DNA containing directional cDNA inserts is enriched for the target sequence by hybridization in solution to a biotinylated oligonucleotide probe complementary to the target sequence. The target sequence is based on the tag sequence of the present invention. The hybrids are captured on streptavidin-coated paramagnetic beads. A magnet retrieves the paramagnetic beads from the solution. leaving nonhybridized single-stranded DNAs behind. Subsequently, the captured single-stranded DNA target is released from the biotinylated oligonucleotide. After release, the cDNA clone is further enriched by using a nonbiotinylated target oligonucleotide to specifically prime conversion of the single-stranded target to double-stranded DNA. Following transformation and plating, typically 20% to 100% of the colonies represent the cDNA clone of interest. To identify the desired cDNA clone, the colonies may be screened by colony hybridization using the ³²P-labeled oligonucleotide as described above for solution hybridization, or alternatively by DNA sequencing and alignment of all sequences obtained from numerous clones to determine a consensus sequence.

4) Isolation of cDNAs from a Library by Probing with the SAGE Transcript or Tag

Classical methods of constructing cDNA libraries are taught in Sambrook et al., supra. Recent procedures described in Velculescu et al. (1997) Science 270:484) can be employed to construct an expression cDNA library cloned into the ZAP Express vector. A ZAP Express cDNA synthesis kit is available from Stratagene is used accordingly to the manufacturer's protocol. Plates containing 250 to 2000 plaques are hybridized as described in Rupert et al. (1988) Mol. Cell. Bio. 8:3104 to oligonucleotide probes with the same conditions previously described for standard probes except that the hybridization temperature is reduced to room temperature. Washes are performed in 6× standard-saline-citrate 0.1% SDS for 30 minutes at room temperature. The probes are labeled with ³²P-ATP through use of T4 polynucleotide kinase.

5) Identification of Known Genes or ESTs

In addition, databases exist that reduce the complexity of ESTs by assembling contiguous EST sequences into tentative genes. For example, TIGR has assembled human ESTs into a database called THC for tentative human consensus sequences. The THC database allows for a more definitive assignment compared to ESTs alone. Software programs exist (TIGR assembler and TIGEM EST assembly machine and contig assembly program (see Huang, X. (1996) Genomics 33:21-23)) that allow for assembling ESTs into contiguous sequences from any organism.

Isolation, Culturing and Expansion of APCs Including Dendritic Cells

Various methods to isolate and characterize APCs including DCs have been known in the art. At least two methods have been used for the generation of human dendritic cells from hematopoietic precursor cells in peripheral blood or bone marrow. One approach cultures the monocytes in GM-CSF and IL-4 as described below, where the immature portion was used for SAGE while an additional portion was treated with TNF-α for 36 hours to encourage their maturation. This method was followed for the isolation and culturing of the cell line identified herein as TNF-α matured dendritic cells.

The other method makes use of the more abundant CD34 precursor population, such as adherent peripheral blood monocytes, and stimulate them with GM-CSF plus IL-4 (see, for example, Sallusto et al. (1994), supra).

In other aspects of the invention, the methods described in Romani et al. (1996), supra or Bender et al. (1996), supra are used to generate both immature and mature dendritic cells from the peripheral blood mononuclear cells (PBMC) of a mammal, such as a murine, simian or human. Briefly, isolated PBMC are pre-treated to deplete T- and B-cells by means of an immunomagnetic technique. Lymphocyte-depleted PBMC are then cultured for 7 days in RPMI medium, supplemented with 1% autologous human plasma and GM-CSF/IL-4, to generate dendritic cells. On day 7, non-adherent cells are harvested for further processing.

The dendritic cells derived from PBMC in the presence of GM-CSF and IL-4 are immature, in that they can lose den Iritic cell properties and revert back to macrophage cell fate if the cytokine stimuli are removed from the culture. A population of dendritic cells having these features were used to isolate the transcripts of immature dendritic cells. The dendritic cells in an immature state are very effective in processing native protein antigens for the MHC class II restricted pathway (Romani et al. (1989) J. Exp. Med 169:1169.)

Further maturation is accomplished by culturing in conditioned medium or treating with LPS or TNF-α or CD40 ligand for 3 days in a macrophage-conditioned medium (CM), which contains the necessary maturation factors. Mature dendritie cells are less able to capture new proteins for presentation but are much better at stimulating resting T cells (both CD4⁺ and CD8⁻) to grow and differentiate.

Mature dendritic cells can be identified by their change in morphology, such as the formation of more motile cytoplasmic processes; by the presence of at least one of the following markers: CD83, CD68, HLA-DR or CD86; or by the loss of Fc receptors such as CD115 (reviewed in Steinman (1991) Ann. Rev. Immunol. 9:271.)

Computational Analysis

This tags identified and claimed herein and populations thereof can be used in further computational analysis. The sequences and expression profiles of this invention are stored in any functionally relevant program, e.g., in Compare Report using the SAGE software (available through Dr. Ken Kinzler at Johns Hopkins University). The Compare Report provides a tabulation of the polynucleotide sequences and their abundance for the samples normalized to a defined number of polynucleotides per library (say 25,000). This information can be imported into MS-ACCESS either directly or via copying the data into an Excel spreadsheet first and then from there into MS-ACCESS for additional manipulations. Other programs such as SYBASE or Oracle that permit the comparison of polynucleotide numbers could be used as alternatives to MS-ACCESS. Enhancements to the software can be designed to incorporate these additional functions. These functions consist in standard Boolean, algebraic, and text search operations, applied in various combinations to reduce a large input set of polynucleotides to a manageable subset of polynucleotides of specifically defined interest.

Sequence information and abundance from a test sample or cell also is input into the functionally relevant program.

The researcher may create groups containing one or more project(s) by combining the counts of specific polynucleotides within a group (e.g., GroupNormal=Normal1+Normal2, GroupTumor=PrimaryTumorl+TumorCellLine). Additional characteristic values are also calculated for each tag in the group (e.g., average count, minimum count, maximum count). The researcher may calculate individual tag count ratios between groups, for example the ratio of the average GroupNormal count to the average GroupTumor count for each polynucleotide. The researcher may calculate a statistical measure of the significance of observed differences in tag counts between groups.

To identify the polynucleotides within MS-ACCESS, a query to sort polynucleotide tags based on their abundance in the sample cells is run. The output from the Query report lists specific polynucleotides (by sequence) that fit the sorting criteria and their abundance in the various sample cells.

The sorting is based on the principle that the gene product of interest (and hence the corresponding polynucleotide) is more abundant in the samples that prominently exhibit the chosen phenotype than in samples that do not exhibit the phenotype.

For example, one may query to identify polynucleotides that are present at a level of 10 tags when the total number of tags per library has been normalized to a defined number in the reference sample against one or more test samples. The results of the search might reveal that 5 different polynucleotides fit the sorting criteria, hence there are 5 candidates genes to be tested to determine whether they confer the phenotype when transferred into samples that do not have the phenotype.

The more stringent the sorting criteria, the more efficient the sorting should be. Thus if one asked for polynucleotides that are at 5 tags when the total number of tags per library has been normalized to a defined number in the reference sample and less than 5 in the test sample, a large number of candidates would be generated. However, if one can increase the differential because the samples manifest extremes of the phenotype (say >10 in the test sample and <1 in the one or more reference samples) this restricts the number of candidates that will be identified.

Prior knowledge of what amount of gene product (hence abundance of polynucleotides) is required to confer the phenotype is not essential as one can arbitrarily select a set of sorting parameters, run the data analysis, and identify and test candidates. If the desired candidate is not found the stringency of the sorting criteria can be reduced (i.e. reduce the differential) and the new candidates that are found can be tested. Iterative cycles of sorting and testing candidates should eventually culminate in the successful recovery of the desired candidate.

Knowledge of what amount of gene product (hence abundance of polynucleotide) is required to confer the phenotype will permit the rationale use of stringent sorting criteria and greatly accelerate the search process as the desired gene may be captured within a handful of candidates Establishing what amount of gene product is required to confer a specific phenotype will be dependent on the specific phenotype in question and the sensitivity of assays that measure that phenotype.

Accordingly, one enters the individual polynucleotide sequences from the Query report into the program to determine if there is a match with any known genes or whether they are potentially novel (no match=NM).

One then retrieves cDNAs corresponding to specific sequences from the Query Report and test them individually in an appropriate biological assay to determine if they confer the phenotype. Of the candidates that correspond to known genes, it is a relatively easy task to obtain complementary DNAs for these candidates and test them individually to determine if they confer the specific phenotype in question when transferred into cells that do not exhibit the phenotype. If none of the known genes confer the phenotype, retrieve the cDNAs corresponding to the No Match sequences of the Query Report by PCR cloning and test the novel cDNAs individually for their ability to confer the phenotype. If the assumptions made up to this point are sound (i.e., a single gene product can confer the phenotype; the sorting criteria are not too stringent so as to exclude the desired candidate) then a cDNA corresponding to one of the candidates of the Query Report will be found to confer the phenotype and the search is over. If however none of the candidates are found to confer the phenotype then one may need to reduce the stringency of the sorting parameters to “cast a wider net” and capture more candidates to be tested as above.

In one embodiment, the polynucleotide or gene sequence can also be compared to a sequence database, for example, using a computer method to match a sample sequence with known sequences. Sequence identity can be determined by a sequence comparison using, i.e., sequence alignment programs that are known in the art, such as those described in CURRENT PROTOCOLS IN MOLECULAR BIOLOGY (F. M. Ausubel et al-, eds., 1987) Supplement 30, section 7.7.18, Table 7.7.1. A preferred alignment program is ALIGN Plus (Scientific and Educational Software, Pennsylvania), preferably using default parameters, which are as follows: mismatch=2; open gap=0; and extend gap=2. Another preferred program is the BLAST program for alignment of two nucleotide sequences, using default parameters as follows: open gap=50; extension gap−2 penalties; gap×dropoff 0; expect=10; word size=11. The BLAST program is available at the following Internet address: http://www.ncbi.nlm.nih.gov. Alternatively, hybridization under conditions of high, moderate and low stringency can also indicate degree of sequence identity.

Vaccines for Cancer Treatment and Prevention

In one embodiment, the present invention comprises vaccines for cancer treatment. Recent advances in vaccine adjuvants provide effective means of administering peptides so that they impact maximally on the immune system. Del-Giudice (1994) Experientia 50:1061-1066. A polynucleotide of this invention can be administered in alone or in combination with a polynucleotide encoding an antigenic peptide as a cancer vaccine. The polynucleotide can be administered as naked DNA or alternatively, in expression vectors. Therapy can be enhanced by coadministration of cytokine and/or co-stimulatory molecules which in turn, can be administered as proteins or the polynucleotides encoding the proteins.

Host Cells Comprising Antigenic Peptides of the Invention

The invention further provides isolated host cells comprising the polynucleotide of the invention. In some embodiments, these host cells present one or more peptides of the invention on the surface of the cell in the context of an MHC molecule, i.e., a antigenic peptide of the invention is bound to a cell surface MHC molecule such that the peptide can be recognized by an immune effector cell. Isolated host cells which present the polypeptides of this invention in the context of MHC molecules are further useful to expand and isolate a population of educated, antigen-specific immune effector cells. The immune effector cells, e.g., cytotoxic T lymphocytes, are produced by culturing naive immune effector cells with antigen-presenting cells cells which present the polypeptides in the context of MHC molecules on the surface of the APCs. The population can be purified using methods known in the art, e.g., FACS analysis or FICOL™ gradient. The methods to generate and culture the immune effector cells as well as the populations produced thereby also are the inventors' contribution and invention. Pharmaceutical compositions comprising the cells and pharmaceutically acceptable carriers are useful in adoptive immunotherapy. Prior to administration in vivo, the immune effector cells are screened in vitro for their ability to lyse melanoma tumor cells.

Gene Transfer

Vectors Useful in Genetic Modification

In one embodiment, the present invention provides methods of eliciting efficient antigen-specific immune response in a subject by introducing to the subject recombinant polynucleotides encoding antigenic peptides alone or in combination with immunostimulatory factors. Methods and materials for gene transfer are known in the art, including, for example, viral mediated gene transfer, lipofection, transformation, transfection and transduction. The polynucleotides encoding the immunostimulatory factor and target antigenic peptide can be introduced ex vivo into a host cell, for example, dendritic cells. The genetically modified host cells can be introduced as a cell-based vaccine into the target subject. Alternatively, the polynucleotides encoding the immunostimulatory factor and target antigenic peptide can be introduced directly into the subject in the form of gene-based vaccine.

Various viral infection techniques have been developed which utilize recombinant viral vectors for gene delivery, and constitute preferred approaches to the present invention. The viral vectors which have been used in gene transfer include, but not limited to, viral sequences derived from simian virus 40 (SV40), adenovirus, adeno-associated virus (AAV), and retroviruses.

Vector Transduction of Cells such as APCs

APCs can be transduced with viral vectors encoding a relevant polypeptides. The most common viral vectors include recombinant poxviruses such as vaccinia and fowlpox virus (Bronte et al. (1997) Proc. Natl. Acad. Sci. USA 94:3183-3188; Kim et al. (1997) J. Immunother. 20:276-286) and, preferentially, adenovirus (Arthur et al. (1997) J. Immunol. 159:1393-1403; Wan et al. (1997) Human Gene Therapy 8:1355-1363; Huang et al. (1995) J. Virol. 69:2257-2263). Retrovirus also may be used for transduction of human APCs (Marin et al. (1996) J. Virol. 70:2957-2962).

In vitro or ex vivo exposure of human DCs to adenovirus (Ad) vector at a multiplicity of infection (MOI) of 500 for 16-24 h in a minimal volume of serum-free medium reliably gives rise to foreign polynucleotide expression in 90-100% of DCs. The efficiency of transduction of DCs or other APCs can be assessed by immunofluorescence using fluorescent antibodies specific for the tumor antigen being expressed (Kim et al. (1997) J. Immunother. 20:276-286). Alternatively, the antibodies can be conjugated to an enzyme (e.g. HRP) giving rise to a colored product upon reaction with the substrate. The actual amount of antigenic polypeptides being expressed by the APCs can be evaluated by ELISA.

In vivo transduction of DCs, or other APCs, can be accomplished by administration of Ad (or other viral vectors) via different routes including intravenous, intramuscular, intranasal, intraperitoneal or cutaneous delivery. The preferred method is cutaneous delivery of Ad vector at multiple sites using a total dose of approximately 1×10¹⁰-1×10¹² i.u. Levels of in vivo transduction can be. roughly assessed by co-staining with antibodies directed against APC marker(s) and the antigen being expressed. The staining procedure can be carried out on biopsy samples from the site of administration or on cells from draining lymph nodes or other organs where APCs (in particular DCs) may have migrated (Condon et al. (1996) Nature Med. 2:1122-1128; Wan et al. (1997) Human Gene Therapy 8:1355-1363). The amount of antigen being expressed at the site of injection or in other organs where transduced APCs may have migrated can be evaluated by ELISA on tissue homogenates.

Although viral gene delivery is more efficient, DCs can also be transduced in vitro/ex vivo by non-viral gene delivery methods such as electroporation, calcium phosphate precipitation or cationic lipid/plasmid DNA complexes (Arthur et al. (1997) Cancer Gene Therapy 4:17-25). Transduced APCs can subsequently be administered to the host via an intravenous. Subcutaneous, intranasal, intramuscular or intraperitoneal route of delivery.

In vivo transduction of DCs, or other APCs, can potentially be accomplished by administration of cationic lipid/plasl lid DNA complexes delivered via the intravenous, intramuscular, intranasal, intraperitoneal or cutaneous route of administration. Gene gun delivery or injection of naked plasmid DNA into the skin also leads to transduction of DCs (Condon et al. (1996) Nature Med. 2:1122-1128 and Raz et al. (1994) Proc. Natl. Acad. Sci. USA 91:9519-9523). Intramuscular delivery of plasmid DNA may also be used for immunization (Rosato et al. (1997) Human Gene Therapy 8:1451-1458.

The transduction efficiency and levels of foreign polynucleotide expression can be assessed as described above for viral vectors.

Administration of Cell-Based Vaccine to Subject

Genetically modified cells can subsequently be administered to the host subject via various routes, including, for example, intravenous infusion, subcutaneous injection, intranasal, intramuscular or intraperitoneal delivery. The cells containing the recombinant polynucleotides may be used to confer immunity to individuals. Administration in vivo can be effected in one dose, continuously or intermittently throughout the course of treatment. Methods of determining the most effective means and dosage of administration are well known to those of skill in the art and will vary with the composition used for therapy, the purpose of the therapy, the target cell being treated, and the subject being treated. Single or multiple administrations can be carried out with the dose level and pattern being selected by the treating physician.

Adoptive Immunotherapy Methods

The expanded populations of antigen-specific immune effector cells and APCs presenting antigens find use in adoptive immunotherapy regimes.

Adoptive immunotherapy methods involve, in one aspect, administering to a subject a substantially pure population of educated, antigen-specific immune effector cells made by culturing naive immune effector cells with APCs as described above. In some embodiments, the APCs are dendritic cells.

In one embodiment, the adoptive immunotherapy methods described herein are autologous. In this case, the APCs are made using parental cells isolated from a single subject The expanded population also employs T cells isolated from that subject. Finally, the expanded population of antigen-specific cells is administered to the same patient.

In a further embodiment, APCs or immune effector cells are administered with an effective amount of a stimulatory cytokine, such as IL-2 or a costimulatory molecule.

Immune Effector Cells

The present invention makes use of the above-described antigen-presenting matrices, including APCs, to stimulate production of an enriched population of antigen-specific immune effector cells. Accordingly, the present invention provides a population of cells enriched in educated, antigen-specific immune effector cells, specific for an antigenic peptide of the invention. These cells can cross-react with (bind specifically to) antigenic determinants (epitopes) on natural (endogenous) antigens. In some embodiments, the natural antigen is on the surface of tumor cells and the educated, antigen-specific immune effector cells of the invention suppress growth of the tumor cells. When APCs are used, the antigen-specific immune effector cells are expanded at the expense of the APCs, which die in the culture. The process by which naive immune effector cells become educated by other cells is described essentially in Coulie (1997) Molec. Med. Today 3:261-268.

An effector cell population suitable for use in the methods of the present invention can be autogeneic or allogeneic, preferably autogeneic. When effector cells are allogeneic, preferably the cells are depleted of alloreactive cells before use. This can be accomplished by any known means, including, for example, by mixing the allogeneic effector cells and a recipient cell population and incubating them for a suitable time, then depleting CD69′ cells, or inactivating alloreactive cells, or inducing anergy in the alloreactive cell population.

Hybrid immune effector cells can also be used. Immune effector cell hybrids are known in the art and have been described in various publications. See, for example, International Patent Application Nos. WO 98/46785; and WO 95/16775.

Although the foregoing invention has been described in some detail by way of illustration and example for purposes of clarty of understanding, it will be apparent to those skilled in the art that certain changes and modifications will be practiced. Therefore, the description and examples should not be construed as limiting the scope of the invention, which is delineated by the appended claims.

### EXAMPLE 1

The Table illustrates a series of mRNAs, both known and unknown, that were found by SAGE analysis to be differentially expressed in monocyte-derived dendritic cells as compared to monocytes. SAGE analysis revealed for instance that the chemokines PARC and TARC that can recruit activated T cells are differentially expressed by monocyte-derived immature dendritic cells (prepared by culturing PBMC derived monocytes in GM-CSF and IL4). Other immunostimulatory factors to mention include: monocyte chemotactic protein-4 (MCP-4) [Berkhout et al., (1997) Journal of Biological Chemistry 272(26): 16404-16413] U46767; macrophage-derived chemokine (MDC) [Godiska et al., (1997) Journal of Experimental Medicine 185(9): 1595-1604] U83171; ecalectin [Matsumoto et al., (1998) Journal of Biological Chemistry 273(27): 16976-16984], AB005894; and monocyte chemotactic protein-2 (MCP-2) [Proost et al., 1996 Journla of Leukocyte Biology 59(1): 67-74] Y10802. The fact that dendritic cells produce abundant levels of these chemokines has not been reported previously. The genes encoding these chemokines could be linked to the gene or genes encoding tumor antigens in a DNA based vaccine to ensure that APCs transduced with the vaccine will produce and process not only the tumor antigen, but also the stimulatory chemokines. Any cDNA or gene encoding any of the mRNAs or combination of mRNAs identified by differential gene expression analysis (such as SAGE) as being differentially expressed in immature dendritic cells as shown in Table I could be linked to a tumor antigen gene or genes to prepare superior vaccines. Similarly, any cDNA or gene encoding any mRNA or combination of mRNAs identified by differential gene expression analysis (such as SAGE) as being differentially expressed in mature dendritic cells could be linked to a tumor antigen gene or genes to prepare superior vaccines.

### EXAMPLE 2

The genes encoding mRNAs that are differentially expressed in either immature or mature dendritic cells are apt to be regulated by specific transcription factors. Thus, an alternative to delivering the gene encoding an mRNA identified as being differentially expressed in either mature or immature dendritic cells by comparative gene expression analysis (such as SAGE) would be to deliver a gene or genes that encode transcription factors (either naturally occurring or engineered via recombinant DNA technology) that can transactivate the expression of the endogenous gene that encodes the differentially expressed mRNA. Thus, the present invention also pertains to vaccines in which a gene or genes encoding tumor antigens is linked to genes encoding transcription factors or transactivators that can upregulate the expression of mRNAs identified as being differentially expressed in either mature or immature dendritic cells by comparative gene expression analysis.

### EXAMPLE 3

Differential gene expression analysis would also be expected to reveal genes encoding cell surface proteins that are preferentially expressed by either immature or mature dendritic cells as compared to monocyte precursors. These cell surface molecules may play a pivotal role in the function of dendritic cells by acting as co-stimulatory signals or modulators of DC function or migration. Naturally occurring ligands specific for these cell surface molecules or recombinant proteins (such as an antibody) generated to have specificity for these cell surface molecules might be expected to interact with the cell surface protein to stimulate the function of the dendritic cells or foster the maintenance of an activated state or stimulate the migration of dendritic cells to sites rich in T cells. Thus, the present invention relates to vaccines in which a gene or genes encoding tumor antigen or antigens is linked to a gene or genes encoding secreted proteins that have the capacity to bind to and modulate the activity of cell surface proteins identified as being differentially expressed in either mature or immature dendritic cells by comparative gene expression analysis (such as SAGE). In this manner, a novel autocrine loop is established whereby a genetically modified APC produces a ligand that is secreted from the APC where it can bind to cell surface receptors on that cell and stimulate the genetically modified dendritic cell.

Although the foregoing invention has been described in some detail by way of illustration and example for purposes of clarity of understanding, it will be apparent to those skilled in the art that certain changes and modifications will be practiced. Therefore, the description and examples should not be construed as limiting the scope of the invention, which is delineated by the appended claims.

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

1. A polynucleotide comprising of a first polynucleotide comprising encoding an immunostimulatory factor that is differentially expressed in an antigen presenting cell and comprising or corresponding to a tag shown in Table 1 or its complement, wherein the first polynucleotide encodes a factor selected from the group consisting of PARC, TARC, monocyte chemoattractant protein-4 (MDP-4), MDC, escalatin, MCP-2 or biologically active fragments thereof.

2. The polynucleotide of claim 1 further comprising a first and second promoter, wherein the first and second polynucleotides are under the transcriptional control of the first and second promoters, respectively.

3. The polynucleotide of claim 1 further comprising a first and second promoter, wherein the first and second polynucleotides are under the transcriptional control of the single promoter.

4. A gene delivery vehicle comprising a polynucleotide of claim 1.

5. A host cell that comprises a polynucleotide of claim 1.

6. An array of probes comprising a polynucleotide of claim 1 bound to a chip.

7. A polynucleotide comprising a first polynucleotide comprising encoding an immunostimulatory factor that is differentially expressed in an antigen presenting cell and comprising or corresponding to a tag shown in Table 1 and a second polynucleotide that modulates the expression of the first polynucleotide, wherein the first polynucleotide encodes PARC, monocyte chemoattractant protein-4 (MDP-4), MDC, escalectin, MCP-2 or biologically active fragments thereof.

8. A polynucleotide of claim 7, wherein said second polynucleotide modulates the expression of a third polynucleotide which encodes an immunostimulatory factor that is differentially expressed in an antigen presenting cell, wherein the third polynucleotide comprises or corresponds to a tag shown in Table 1.

9. A gene delivery vehicle comprising the polynucleotides of claim 7.

10. A host cell comprising the polynucleotides of claim 7.

11. A method for inducing an immune response in a subject comprising administering an effective amount of the polynucleotide of claim 1, to the subject.

12. A method of modulating the genotype of an antigen presenting cell, comprising introducing into the cell a polynucleotide of claim 1.
